(19) United States
(12) Patent Application Publication
(10) Pub. No.: US 2026/0130347 A1
(43) Pub. Date: May 14, 2026

(54) TRANSGENIC ANIMALS AND RECOMBINANT HOST CELLS COMPRISING SPECIFIC GENE VARIANTS AND METHODS OF CREATING SAME

(71) Applicant: Colossal Biosciences Inc., Austin, TX (US)

(72) Inventors: Scott BARISH, Austin, TX (US); Kathleen Morrill PIROVICH, Austin, TX (US); Greg GEDMAN, Austin, TX (US); Sven BOCKLANDT, Austin, TX (US); Ben E. LAMM, Austin, TX (US); Beth SHAPIRO, Austin, TX (US)

(21) Appl. No.: 19/208,979

(22) Filed: May 15, 2025

Related U.S. Application Data
(63) Continuation of application No. PCT/US2024/061594, filed on Dec. 20, 2024.
(60) Provisional application No. 63/612,468, filed on Dec. 20, 2023.
(60) Provisional application No. 63/612,477, filed on Dec. 20, 2023.

(51) Int. Cl. A01K 67/0276; C07K 14/47; C12N 5/077

(52) U.S. Cl. CPC A01K 67/0276; C07K 14/47; C12N 5/0656; A01K 2217/054; A01K 2217/077; A01K 2227/101; C12N 2510/00

(57) ABSTRACT

Provided herein are isolated nucleic acids, vectors, recombinant cells, and transgenic animals capable of expressing at least one or more bluebuck gene variants associated with gray/blue coat color and/or melanogenesis and/or altered facial pattern. Also provided are methods of making recombinant cells and/or transgenic animals that comprise at least one bluebuck (Hippotragus leucophaeus) gene variant associated with gray/blue coat color and/or melanogenesis and/or altered facial pattern.

### CROSS REFERENCE TO RELATED APPLICATIONS

This application is a continuation application of International Patent Application No. PCT/US2024/061594, filed Dec. 20, 2024, which claims priority to U.S. Provisional Application No. 63/612,468, filed Dec. 20, 2023, and U.S. Provisional Application No. 63/612,477, filed Dec. 20, 2023. Each disclosure is herein incorporated by reference in its entirety.

### FIELD OF THE INVENTION

The present disclosure generally relates to gene edited, and/or reprogrammed mammalian cells and uses thereof.

### REFERENCE TO SEQUENCE LISTING SUBMITTING ELECTRONICALLY

This application contains a sequence listing, which is submitted electronically. The contents of the electronic sequence listing (069296.15US3 Sequence Lisitng.xml; size: 15,137,605 bytes; and creation date of May 15, 2025) is herein incorporated by reference in its entirety.

### BACKGROUND OF THE INVENTION

Bluebucks or blue antelopes (Hippotragus leucophaeus) were a species of antelope that lived in South Africa until around 1800. Bluebucks were smaller than the roan antelope and sable antelope and were sometimes considered a subspecies of the roan antelope, but genetic studies have determined that it was a distinct species. The coat of the bluebuck was a uniform bluish-gray with a pale whitish belly. The forehead was brown and darker than the face. The mane of the bluebuck was not as developed as the mane of the roan and sable antelopes, and the ears of the bluebuck were shorter and blunter and not tipped with black. The bluebuck had a darker tail tuft and smaller teeth than those of the roan and sable antelopes.

The bluebuck was a grazer animal and was believed to have calved where rainfall would peak, as there would be an abundance of available grasses to eat. The bluebuck was confined to the southwestern Cape when encountered by the Europeans, but fossil evidence and rock paintings show that the bluebuck originally had a larger distribution. Bluebucks were common across South Africa, but by the time Europeans encountered the bluebuck in the 17th century, it was already uncommon, perhaps due to its preferred grassland habitat having been reduced in size mainly along the southern coast of South Africa.

The generation of bluebuck antelopes and the restoration of a population of this extinct species can have a wide array of benefits, including, but not limited to, conservation and de-extinction efforts. In addition, generation of bluebuck antelopes preserve species diversity to combat climate change effects.

Accordingly, there is a need to restore bluebucks. This disclosure provides methods to generate bluebuck cells and transgenic bluebuck animals.

### BRIEF SUMMARY OF THE INVENTION

Provided herein are transgenic animals comprising at least one bluebuck (Hippotragus leucophaeus) gene variant associated with a gray/blue coat color, melanogenesis, and/or an altered facial pattern and methods of creating such transgenic animals.

Provided herein are transgenic animals comprising one or more bluebuck (Hippotragus leucophaeus) gene variants associated with gray/blue coat color and/or melanogenesis. The transgenic animals can, for example comprise one or more bluebuck gene variants associated with gray/blue coat color selected from an ATP Binding Cassette Subfamily B Member 6 (LAN Blood Group) (ABCB6) variant, an Angiotensin Converting Enzyme 2 (ACE2) variant, an Adaptor Related Protein Complex 3 Subunit Beta 1 (AP3B1) variant, an Adaptor Related Protein Complex 3 Subunit Delta 1 (AP3D1) variant, an Agouti Signaling Protein (ASIP) variant, an ATPase Copper Transporting Alpha (ATP7A) variant, a BCL2 Apoptosis Regulator (BCL2) variant, a Biogenesis Of Lysosomal Organelles Complex 1 Subunit 2 (BLOC1S2) variant, a Biogenesis Of Lysosomal Organelles Complex 1 Subunit 3 (BLOC1S3) variant, a Biogenesis Of Lysosomal Organelles Complex 1 Subunit 4 (BLOC1S4) variant, a Biogenesis Of Lysosomal Organelles Complex 1 Subunit 5 (BLOC1S5) variant, a B-Raf Proto-Oncogene, Serine/Threonine Kinase (BRAF) variant, a Dopachrome Tautomerase (DCT) variant, a Dynactin Subunit 1 (DCTN1) variant, a Dynactin Subunit 2 (DCTN2) variant, a Dedicator Of Cytokinesis 7 (DOCK7) variant, a Dual Serine/Threonine And Tyrosine Protein Kinase (DSTYK) variant, a Dystrobrevin Binding Protein 1 (DTNBP1) variant, an Embryonic Ectoderm Development (EED) variant, a FIG. 4 Phosphoinositide 5-Phosphatase (FIG. 4) variant, a Gamma-Glutamyltransferase 1 (GGT1) variant, a G Protein-Coupled Receptor 143 (GPR143) variant, a HPS1 Biogenesis Of Lysosomal Organelles Complex 3 Subunit 1 (HPS1) variant, a HPS3 Biogenesis Of Lysosomal Organelles Complex 2 Subunit 1 (HPS3) variant, a HPS4 Biogenesis Of Lysosomal Organelles Complex 3 Subunit 2 (HPS4) variant, a HPS5 Biogenesis Of Lysosomal Organelles Complex 2 Subunit 2 (HPS5) variant, a HPS6 Biogenesis Of Lysosomal Organelles Complex 2 Subunit 3 (HPS6) variant, a Lysosomal Trafficking Regulator (LYST) variant, a Membrane Bound Transcription Factor Peptidase, Site 1 (MBTPS1) variant, a Mucolipin TRP Cation Channel 3 (MCOLN3) variant, a Major Facilitator Superfamily Domain Containing 12 (MFSD12) variant, a Melanocyte Inducing Transcription Factor (MITF) variant, a Muskelin 1 (MKLN1) variant, a Melan-A (MLANA) variant, a Melanophilin (MPLH) variant, a Melanoregulin (MREG) variant, a Myosin VA (MYO5A) variant, a Notch Receptor 1 (NOTCH1) variant, a Notch Receptor 2 (NOTCH2) variant, an OCA2 Melanosomal Transmembrane Protein (OCA2) variant, a Phenylalanine Hydroxylase (PAH) variant, a Phosphoinositide Kinase, FYVE-Type Zinc Finger Containing (PIKFYVE) variant, a Premelanosome Protein (PMEL) variant, a RAB27A, Member RAS Oncogene Family (RAB27A) variant, a RAB38, Member RAS Oncogene Family (RAB38) variant, a Rab Geranylgeranyltransferase Subunit Alpha (RABGGTA) variant, a Raf-1 Proto-Oncogene, Serine/Threonine Kinase (RAF1) variant, a Solute Carrier Family 24 Member 5 (SLC24A5) variant, a Solute Carrier Family 45 Member 2 (SLC45A2) variant, a Solute Carrier Family 7 Member 11 (SLC7A11) variant, a Snail Family Transcriptional Repressor 2 (SNAI2) variant, a Sperm Associated Antigen 9 (SPAG9) variant, a Two Pore Segment Channel 2 (TPCN2) variant, a Tyrosinase (TYR) variant, a Tyrosinase Related Protein 1 (TYRP1) variant, a VAC14 Component Of PIKFYVE Complex (VAC14) variant, a VPS33A Core Subunit Of CORVET And HOPS Complexes (VPS33A) variant, and a YKT6 V-SNARE Homolog (YKT6) variant. The transgenic animals can, for example comprise one or more bluebuck gene variants associated with melanogenesis selected from an Adaptor Related Protein Complex 3 Subunit Delta 1 (AP3D1) variant, an Activating Signal Cointegrator 1 Complex Subunit 2 (ASCC2) variant, an ATPase H+ Transporting V0 Subunit A2 (ATP6V0A2) variant, a CCDC53 variant, a CD72 molecule (CD72) variant, a Cyclin Dependent Kinase 12 (CDK12) variant, a Chromodomain Helicase DNA Binding Protein 8 (CHD8) variant, a COMM Domain Containing 5 (COMMD5) variant, a DSCR3 variant, an ElA Binding Protein P300 (EP300) variant, a FIG. 4 Phosphoinositide 5-Phosphatase (FIG. 4) variant, a FRY Like Transcription Coactivator (FRYL) variant, a Histone Deacetylase 4 (HDAC4) variant, a Heterogeneous Nuclear Ribonucleoprotein A2/B1 Like 1 (HNRNPA2B1L1) variant, a Heterogeneous Nuclear Ribonucleoprotein A2/B1 Like 2 (HNRNPA2B1L2) variant, a Heterogeneous Nuclear Ribonucleoprotein A2/B1 Like 3 (HNRNPA2B1L3) variant, a Heterogeneous Nuclear Ribonucleoprotein A2/B1 Like 4 (HNRNPA2B1L4) variant, a HPS3 Biogenesis Of Lysosomal Organelles Complex 2 Subunit 1 (HPS3) variant, a HPS4 Biogenesis Of Lysosomal Organelles Complex 3 Subunit 2 (HPS4) variant, a HPS5 Biogenesis Of Lysosomal Organelles Complex 2 Subunit 2 (HPS5) variant, a HPS6 Biogenesis Of Lysosomal Organelles Complex 2 Subunit 3 (HPS6) variant, an Integrator Complex Subunit 8 (INTS8) variant, a Lysosomal Trafficking Regulator (LYST) variant, a Nuclear Receptor Corepressor 1 (NCOR1) variant, a RAB9A, Member RAS Oncogene Family (RAB9A) variant, a RB Transcriptional Corepressor 1 (RB1) variant, a SR-Related CTD Associated Factor 4 (SCAF4) variant, a SEC31 Homolog A, COPII Coat Complex Component (SEC31A) variant, a Siah E3 Ubiquitin Protein Ligase 1 (SIAH1) variant, a SMG7 Nonsense Mediated MRNA Decay Factor (SMG7) variant, a Sorting Nexin 13 Like 1 (SNX13L1) variant, a Sorting Nexin 13 Like 2 (SNX13L2) variant, a TBC1 Domain Family Member 5 (TBC1D5) variant, and a WD Repeat And FYVE Domain Containing 1 (WDFY1) variant.

Also provided are transgenic animals comprising one or more bluebuck (Hippotragus leucophaeus) gene variants associated with an altered facial pattern, wherein the one or more bluebuck gene variants associated with altered facial pattern are selected from an ADAM Metallopeptidase With Thrombospondin Type 1 Motif 20 (ADAMTS20) variant, a Bone Morphogenetic Protein 4 (BMP4) variant, a Dedicator Of Cytokinesis 7 (DOCK7) variant, an Endothelin 3 (EDN3) variant, an Endothelin Receptor Type B (EDNRB) variant, an ETS Proto-Oncogene 1, Transcription Factor Like 1 (ETSIL1) variant, an ETS Proto-Oncogene 1, Transcription Factor Like 2 (ETS1L2) variant, a Forkhead Box D3 (FOXD3) variant, a Histone Deacetylase 1 (HDAC1) variant, an Interferon Regulatory Factor 1 (IRF1) variant, a KIT Proto-Oncogene, Receptor Tyrosine Kinase (KIT) variant, a KIT Ligand (KITLG) variant, a Mucolipin TRP Cation Channel 3 (MCOLN3) variant, a Melanocyte Inducing Transcription Factor (MITF) variant, a Paired Box 3 (PAX3) variant, a Snail Family Transcriptional Repressor 2 (SNAI2) variant, a SRY-Box Transcription Factor 10 (SOX10) variant, a SRY-Box Transcription Factor 2 (SOX2) variant, a Transient Receptor Potential Cation Channel Subfamily M Member 1 (TRPM1) variant, a Wnt Family Member 3A (WNT3A) variant, and a Zinc Finger Homeobox 4 (ZFHX4) variant.

Also provided are transgenic animals comprising one or more bluebuck (Hippotragus leucophaeus) gene variants associated with gray/blue coat color, wherein the one or more bluebuck gene variants comprise (a) a loss-of-function agouti signaling protein (ASIP) variant; (b) a defensin beta 103 (DEFB103) missense variant; (c) a loss-of-function melanophilin (MLPH) variant; (d) a premelanosome protein (PMEL) missense variant; (e) a loss-of-function lysosomal trafficking regulator (LYST) variant; and/or (f) a loss-of-function solute carrier family 45 member 2 (SLC45A2) variant.

Also provided are methods of creating a transgenic animal comprising a bluebuck gene variant associated with gray/blue coat color and/or an altered melanin biosynthesis pathway of a bluebuck. The methods comprise: (a) obtaining a cell from the animal; (b) introducing into the cell at least one or more bluebuck (Hippotragus leucophaeus) gene variants associated with gray/blue coat color and/or melanogenesis, wherein the one or more bluebuck gene variants associated with gray/blue coat color are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant, and the one or more bluebuck gene variants associated with melanogenesis are selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant; and (c) utilizing the recombinant cell to produce a transgenic animal, wherein the transgenic animal has a gray/blue coat color and/or melanogenesis.

Also provided are methods of creating a transgenic animal comprising a bluebuck gene variant associated with gray/blue coat color. The methods comprise: (a) obtaining a cell from the animal; (b) introducing into the cell at least one bluebuck gene variant selected from: (1) a loss-of-function ASIP variant; (2) a DEFB103 missense variant; (3) a loss-of-function MLPH variant; (4) a PME) missense variant; (5) a loss-of-function LYST variant; and/or (6) a loss-of-function SLC45A2 variant, whereby introducing the at least one bluebuck gene variant produces a recombinant cell; and (c) utilizing the recombinant cell to produce a transgenic animal, wherein the transgenic animal has a gray/blue coat color.

Also provided are methods of creating a transgenic animal comprising a bluebuck gene variant associated with altered facial pattern. The methods comprise: (a) obtaining a cell from the animal; (b) introducing into the cell at least one of a bluebuck gene variant associated with altered facial pattern selected from an ADAMTS20 variant, a BMP4 variant, a DOCK7 variant, an EDN3 variant, an EDNRB variant, an ETSILI variant, an ETS1L2 variant, a FOXD3 variant, a HDAC1 variant, an IRFI variant, a KIT variant, a KITLG variant, a MCOLN3 variant, a MITF variant, a PAX3 variant, a SNAI2 variant, a SOX10 variant, a SOX2 variant, a TRPMI variant, a WNT3A variant, and a ZFHX4 variant; and (c) utilizing the recombinant cell to produce a transgenic animal, wherein the transgenic animal has an altered facial pattern.

In certain embodiments, the transgenic animal comprises 2, 3, 4, 5, 6, 7, 8, 9, 10, 15, 20, 25, 30, 40, 50, 60, 70, 80, 90, 95, 100, 101, 102 (or any number in between) bluebuck gene variants. In certain embodiments, the bluebuck gene variant comprises at least one change in the nucleotide sequence of the gene. The change in the nucleotide sequence can, for example, be a substitution, an insertion, a deletion, or a combination thereof. The substitution, the insertion, the deletion, or a combination thereof can, for example, be in a 5′ untranslated region of the gene, an intron of the gene, an exon of the gene, a 3′ untranslated region of the gene, or a combination thereof. The substitution, the insertion, the deletion, or a combination thereof can, for example, be in a regulatory region of the gene. In other embodiments, the bluebuck gene variant comprises one or more substitutions within the regulatory region and/or within the coding region of the gene as compared to a corresponding reference genome, wherein the corresponding reference genome is the genome of the animal prior to the one or more substitutions.

In certain embodiments, the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof. The bluebuck gene variant associated with gray/blue coat color can, for example, comprise a nucleotide sequence selected from the group consisting of SEQ ID NOs: 147-204, and combinations thereof.

In certain embodiments, the bluebuck gene variant associated with melanogenesis comprises a nucleotide sequence having at least 80%, at least 85%, at least 90%, or at least 95% identity to a nucleotide sequence selected from the group consisting of SEQ ID NOs: 48, 64, 68-72, 120-146, and combinations thereof. The bluebuck gene variant associated with melanogenesis can, for example, comprise a nucleotide sequence selected from the group consisting of SEQ ID NOs: 150, 166, 170-174, 222-248, and combinations thereof.

In certain embodiments, wherein the transgenic animals comprising one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the transgenic animal can further comprise one or more bluebuck gene variants associated with altered facial pattern selected from an ADAMTS20 variant, a BMP4 variant, a DOCK7 variant, an EDN3 variant, an EDNRB variant, an ETS1L1 variant, an ETSIL2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPMI variant, a WNT3A variant, and a ZFHX4 variant.

In certain embodiments, the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof. The bluebuck gene variant associated with altered facial pattern can, for example, comprise a nucleotide sequence selected from the group consisting of SEQ ID NOs: 162, 176, 178, 197, 205-221, and combinations thereof.

In certain embodiments, wherein the transgenic animal comprises one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the transgenic animal can further comprise one or more bluebuck gene variants associated with gray/blue coat color selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant. In certain embodiments, the transgenic animal can further comprise one or more bluebuck gene variants associated with altered facial pattern selected from an ADAMTS20 variant, a BMP4 variant, a DOCK7 variant, an EDN3 variant, an EDNRB variant, an ETSIL1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the transgenic animal fails to express the endogenous homologue of at least 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the transgenic animal is an antelope. In certain embodiments, the transgenic animal is a roan antelope (Hippotragus equinus) or a sable antelope (Hippotragus niger).

Also provided are recombinant host cells comprising one or more bluebuck (Hippotragus leucophaeus) gene variants associated with gray/blue coat color and/or melanogenesis. In certain embodiments, the recombinant host cells comprise one or more bluebuck gene variants associated with gray/blue coat color selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant. In certain embodiments, the recombinant host cells comprise one or more bluebuck gene variants associated with melanogenesis selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant.

Also provided are recombinant host cells comprising one or more bluebuck (Hippotragus leucophaeus) gene variants associated with altered facial pattern. In certain embodiments, the recombinant host cells comprise one or more bluebuck gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Also provided are recombinant host cells comprising one or more bluebuck (Hippotragus leucophaeus) gene variants associated with gray/blue coat color. The recombinant host cells comprise one or more bluebuck gene variants selected from (a) a loss-of-function ASIP variant; (b) a DEFB103 missense variant; (c) a loss-of-function MLPH variant; (d) a PMEL missense variant; (e) a loss-of-function LYST variant; and/or (d) a loss-of-function SLC45A2 variant.

In certain embodiments, the recombinant host cell comprises 2, 3, 4, 5, 6, 7, 8, 9, 10, 15, 20, 25, 30, 40, 50, 60, 70, 80, 90, 95, 100, 101, 102 (or any number in between) bluebuck gene variants. In certain embodiments, the bluebuck gene variant comprises at least one change in the nucleotide sequence of the gene. The change in the nucleotide sequence can, for example, be a substitution, an insertion, a deletion, or a combination thereof. The substitution, the insertion, the deletion, or a combination thereof can, for example, be in a 5′ untranslated region of the gene, an intron of the gene, an exon of the gene, a 3′ untranslated region of the gene, or a combination thereof. The substitution, the insertion, the deletion, or a combination thereof can, for example, be in a regulatory region of the gene. In other embodiments, the bluebuck gene variant comprises one or more substitutions within the regulatory region and/or within the coding region of the gene as compared to a corresponding reference genome, wherein the corresponding reference genome is the genome of the recombinant host cell prior to the one or more substitutions.

In certain embodiments, the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof. The bluebuck gene variant associated with gray/blue coat color can, for example, comprise a nucleotide sequence selected from the group consisting of SEQ ID NOs: 147-204, and combinations thereof.

In certain embodiments, the bluebuck gene variant associated with melanogenesis comprises a nucleotide sequence having at least 80%, at least 85%, at least 90%, or at least 95% identity to a nucleotide sequence selected from the group consisting of SEQ ID NOs: 48, 64, 68-72, 120-146, and combinations thereof. The bluebuck gene variant associated with melanogenesis can, for example, comprise a nucleotide sequence selected from the group consisting of SEQ ID NOs: 150, 166, 170-174, 222-248, and combinations thereof.

In certain embodiments, in recombinant host cells comprising one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the recombinant host cell can further comprise a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof. The bluebuck gene variant associated with altered facial pattern can, for example, comprise a nucleotide sequence selected from the group consisting of SEQ ID NOs: 162, 176, 178, 197, 205-221, and combinations thereof.

In certain embodiments, in recombinant host cells comprising one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the recombinant host cell can further comprise one or more bluebuck gene variants associated with gray/blue coat color selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant. In certain embodiments, the recombinant host cell can further comprise a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the recombinant host cell fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the recombinant host cell fails to express the endogenous homologue of at least 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the recombinant host cell is an antelope cell. In certain embodiments, the recombinant host cell is a roan antelope (Hippotragus equinus) cell or a sable antelope (Hippotragus niger) cell.

In certain embodiments, the recombinant host cell is a stem cell. The stem cell can, for example, be selected from an induced stem cell, an embryonic stem (ES) cell, or a mesenchymal stem cell (MSC).

In certain embodiments, the recombinant host cell is a reprogrammed cell.

In certain embodiments, the recombinant host cell is a fibroblast cell or a mesenchymal cell. In certain embodiments, the recombinant host cell is an endothelial progenitor cell (EPC) or a pericyte. The recombinant host cell can, for example, be selected from the group consisting of a nerve cell, a cartilage cell, a bone cell, a muscle cell, a fat cell, and an epidermal cell.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1A and FIG. 1B show the editing efficiency of ASIP in roan fibroblasts. FIG. 1A shows percent of modified reads, indicating successful editing of the ASIP gene. FIG. 1B shows reads with abundance percentage of NGS (next-generation sequencing) reads of ASIP edited cells. Reads show indel generation.

FIG. 2A and FIG. 2B show the editing efficiency of MLPH in roan fibroblasts. FIG. 2A shows percent of modified reads, indicating successful editing of the MLPH gene. FIG. 2B shows reads with abundance percentage of NGS reads of MLPH edited cells. Reads show indel generation.

FIG. 3A and FIG. 3B show the editing efficiency of LYST in roan fibroblasts. FIG. 3A shows percent of modified reads, indicating successful editing of the LYST gene. FIG. 3B shows reads with abundance percentage of NGS reads of LYST edited cells. Reads show indel generation.

FIG. 4A and FIG. 4B show the editing efficiency of SLC45A2 in roan fibroblasts. FIG. 4A shows percent of modified reads, indicating successful editing of the SCL45A2 gene. FIG. 4B reads with abundance percentage of NGS reads of SLC45A2 edited cells. Reads show indel generation.

FIG. 5 shows a comparison of the editing efficiency of MITF locus in roan fibroblasts for 7 guides. Dark gray bars represent percent of edited reads measure by NGS. Light gray bars represent unedited reads. Negative controls show no editing.

FIG. 6 shows a representative image of transfection of GFP-Cas9 RNP (Ribonucleoprotein) in roan fibroblast cells. Dots visible inside of cells indicate successfully transfected GFP-Cas9 24 hours post transfection. Images take on INCUCYTE® (Sartorius, Göttingen, Germany)

FIG. 7 shows NGS reads of monoclonal colony isolated after editing of the LYST gene in roan fibroblast cells. 96.93% of reads show modification of the target locus indicating biallelic editing. Reads also show an eleven base pair deletion, which results in a frameshift of the LYST coding sequence and a loss of function of the gene.

FIG. 8A and FIG. 8B show representative images of a monoclonal colony in a single well of a 96-well plate of roan fibroblast cells isolated by FAC sorting. Image shown in FIG. 8A was taken 5 days after monoclonal isolation; image shown in FIG. 8B was taken 7 days post isolation and shows significant outgrowth indicating successful isolation.

FIG. 9 shows an image of a chimeric gray mouse containing prototype edits to ASIP and MLPH. The mouse was generated from an albino embryo injected with 129 embryonic stem cells containing knockout edits to ASIP and MLPH as a proof of concept for antelope edits. Gray patches demonstrated that these edits can alter coat color from brown to gray.

### DETAILED DESCRIPTION OF THE INVENTION

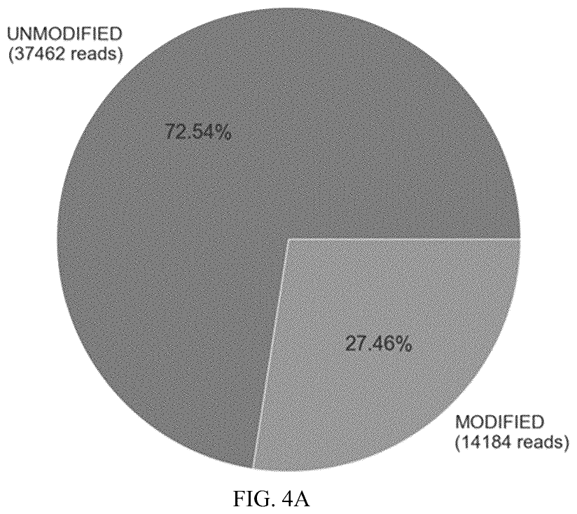

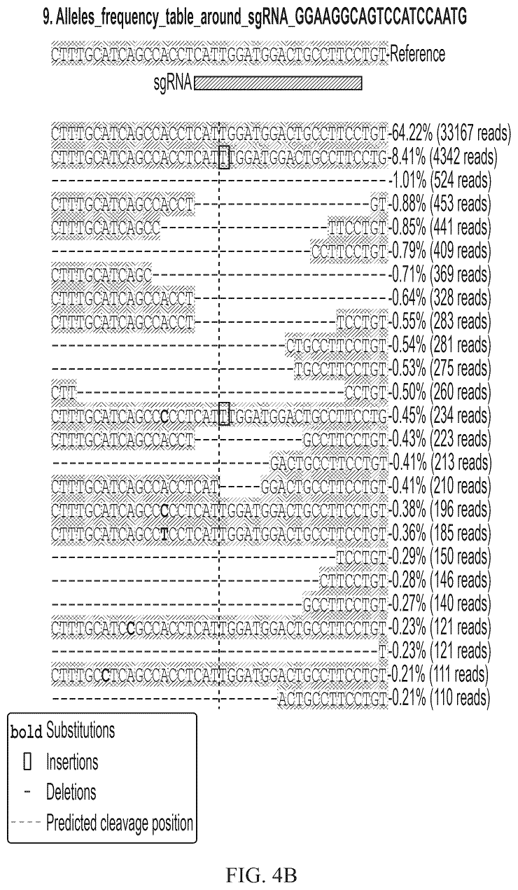

This disclosure is based on the discovery that using only a minimal number of bluebuck (Hippotragus leucophaeus) gene variants it is possible to generate a transgenic animal with the gray/blue coat color and/or melanogenesis and/or altered facial pattern of bluebucks. Accordingly, the disclosure provides transgenic animals, recombinant host cells, and methods of generating transgenic animals by introducing at least one or more bluebuck (Hippotragus leucophaeus) gene variants associated with gray/blue coat color and/or melanogenesis, wherein the one or more bluebuck gene variants associated with gray/blue coat color are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant, and the one or more bluebuck gene variants associated with melanogenesis are selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant. The disclosure also provides transgenic animals, recombinant host cells, and methods of creating transgenic animals by introducing one or more bluebuck gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the present disclosure, such as, for example an isolated vector comprising an isolated nucleic acid of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome. A host cell can be, for example, any type of prokaryotic, eukaryotic, or archaeal cell. In some instances, the host cell is a bacterial cell. In some instances, the host cell is a mammalian cell.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “heterologous nucleic acid” or “heterologous polypeptide” refers to a nucleic acid or a polypeptide whose sequence is not identical to that of another nucleic acid or polypeptide naturally found in the same host cell or the same host. As use herein, the “heterologous nucleic acid” or “heterologous polypeptide” can be heterologous to the bacterial cell and/or the mammalian host.

As used herein, the term “transform” or “transformation” refers to the transfer of a nucleic acid fragment into a host cell, such as a host bacterial cell, resulting in genetically-stable inheritance. Host cells comprising the transformed nucleic acid fragment are referred to as “recombinant” or “transgenic” or “transformed” organisms.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, “gene” refers to a nucleic acid comprising an open reading frame encoding a polypeptide, including both exon and (optionally) intron sequences.

As used herein, a “promoter” is an example of a transcriptional regulatory sequence and is specifically a nucleic acid sequence generally described as the proximal region of a gene located 5′ to the start codon. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

The term “gene product,” as used herein, refers to any product encoded by a nucleic acid sequence. Accordingly, a gene product may, for example, be a primary transcript, a mature transcript, a processed transcript, or a protein or peptide encoded by a transcript. Examples for gene products, accordingly, include mRNAs, rRNAs, hairpin RNAs (e.g., microRNAs, shRNAs, siRNAs, tRNAs), and peptides and proteins, for example, reporter proteins or therapeutic proteins.

As used herein, the term “stem cell” refers to a cell that can self-renew and differentiate to at least one more-differentiated or less developmentally-capable phenotype. The term “stem cell” encompasses stem cell lines, induced stem cells, non-human embryonic stem cells, pluripotent stem cells, multipotent stem cells, amniotic stem cells, placental stem cells, or adult stem cells. An “induced stem cell” is one derived from a non-pluripotent cell induced to a less-differentiated or more developmentally-capable phenotype by introduction of one or more reprogramming factors or genes. As the term is used herein, an induced stem cell need not be pluripotent, but has the capacity to differentiate, under appropriate conditions, to more than one more-highly-differentiated phenotype. It should be understood that the capacity was not present prior to the introduction of reprogramming factors. An induced stem cell will express at least one stem cell marker not expressed by the parent cell prior to introduction of reprogramming factors. In this context, a stem cell marker is exclusive of a factor introduced by reprogramming. An induced pluripotent stem cell, or iPS cell, has the induced capacity to differentiate, under appropriate conditions, to a cell phenotype derived from each of the endoderm, mesoderm, and ectoderm germ layers.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “exogenous” refers to a substance present in a cell that was introduced by the hand of man. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found. Alternatively, “exogenous” can refer to a nucleic acid or a polypeptide that has been introduced by a process involving the hand of man into a biological system such as a cell or organism in which it is found in relatively lower amounts and in which one wishes to increase the amount of the nucleic acid or polypeptide in the cell or organism, e.g., to create ectopic expression or levels.

As used herein, the term “reprogramming genes” or “reprogramming factors” refers to agents or nucleic acid molecules that can induce the reprogramming process in a somatic cell to re-express a less-differentiated, more stem-cell like phenotype. The reprogramming factor can be a nucleic acid, a polypeptide, or a small molecule that promotes a reprogrammed phenotype when introduced to a cell. Non-limiting examples of reprogramming factors include: Oct4 (Octamer binding transcription factor-4), SOX2 (Sex determining region Y)-box 2, Klf4 (Kruppel Like Factor-4), and c-Myc. These are the so-called “classical” or “standard” set of reprogramming factors used to derive, for example, induced pluripotent stem cells. Additional factors that can be considered reprogramming factors when introduced in the process of reprogramming cells to a less differentiated or stem cell phenotype include LIN28+Nanog, Esrrb, Pax5 shRNA, C/EBPa, p53 siRNA, UTF1, DNMT shRNA, Wnt3a, SV40 LT (T), hTERT, small molecule chemical agents including, but not limited to BIX-01294, BayK8644, RG108, AZA, dexamethasone, VPA, TSA, SAHA, PD0325901+CHIR99021 (2i) and A-83-01. In some embodiments, the reprogramming genes or factors are Oct4, Klf4, SOX2, and c-Myc.

As used herein, the terms “dedifferentiation” or “retrodifferentiation” or “reprogramming” refer to a process that generates a cell that re-expresses a less differentiated phenotype than the cell from which it is derived and/or expresses at least one stem cell marker not expressed prior to that process. For example, a terminally-differentiated cell can be dedifferentiated to a multipotent cell. That is, dedifferentiation shifts a cell backward along the differentiation spectrum of totipotent cells to fully differentiated cells. Typically, reversal of the differentiation phenotype of a cell requires artificial manipulation of the cell, for example, by introducing or expressing exogenous polypeptide factors. Reprogramming is not typically observed under native conditions in vivo or in vitro.

As used herein, a “reprogrammed cell” is a cell that has been contacted with one or more reprogramming factors and expresses a less differentiated phenotype than the cell from which it was derived. The reprogrammed cell can also have the capacity to self-renew and will express at least one stem cell marker that was not delivered to the cell as a reprogramming factor. Furthermore, the reprogrammed cell will have the capacity to differentiate into a more-differentiated somatic cell type following differentiation protocols provided herein or described in the art.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, excluding germ cells. Every cell type in the mammalian body-apart from the sperm and ova and the cells from which they are made (gametocytes)—is a somatic cell: internal organs, skin, bones, blood, and connective tissue are all substantially made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

### Nucleic Acids, Vectors, Recombinant Cells, and Transgenic Animals Expressing Bluebuck Specific Variants

The generation of bluebuck antelopes and the restoration of a population of this extinct species can have a wide array of benefits, including, but not limited to, conservation and de-extinction efforts, as the methods used herein can restore an extinct species and preserve species diversity to combat climate change effects. This method can also be used to preserve existing species with blue/gray coat colors and distinctive facial patterns and likely confer beneficial adaptations to habitats, as well as preserve existing environments through rewilding efforts. Additionally, there can be potential economic benefits, including the production of blue fur skins for clothing that may be highly coveted. Finally, as part of the genomic editing methods described herein, novel genomic information about living antelope species Hippotragus niger (roan antelope) and Hippotragus equinus (sable antelope), as well as the extinct bluebuck antelope has been produced. This information can aid in the research efforts around evolutionary biology, speciation, and ecology. Finally, the de-extinction of bluebuck will advance synthetic biology and will serve as a precedent for advanced multiplexed editing in organisms.

The isolated nucleic acids, vectors, recombinant cells, and transgenic animals described herein are based, in part, on the discovery that cells (e.g., Hippotragus niger (roan antelope) and Hippotragus equinus (sable antelope) cells) can be modified to comprise and express alleles or homologues from the bluebuck (e.g., Hippotragus leucophaeus). In particular, viable cells can be gene-edited, whether by transfection, transduction or modification of existing antelope homologues to mimic the bluebuck variants or alleles of the bluebuck genes. In some embodiments, the endogenous homologues of the bluebuck genes are deleted or inactivated. Similar modifications to introduce bluebuck genes can be made to viable cells of other, non-human relatives of the antelope. The bluebuck variants or alleles can modify the phenotype of the gene edited cells. The isolated nucleic acids, vectors, recombinant cells, and transgenic animals described herein provide a synthetic alternative to wildlife products and new tools for understanding genetic diversity and cellular biology in endangered and extinct species of wildlife.

In one aspect, described herein is at least one exogenous nucleic acid sequence encoding a bluebuck gene, or comprising a modification of an endogenous gene to express a bluebuck homologue or variant of the endogenous gene. Of particular interest are genes that are shared by every bluebuck genome sequenced, which are not shared by any antelope genome sequenced. By choosing genes in this manner, effects of individual variation within the group of bluebuck genomes sequenced and variations in antelope genomes (roan and sable) are minimized to focus on those variant sequences that are fully bluebuck. In view of this, as used herein, a “bluebuck gene,” “bluebuck gene variant” or “bluebuck homologue” is a gene encoding a polypeptide that has a sequence encoded by all bluebuck genomes sequenced, and which differs from the homologous polypeptide encoded in all antelope genomes sequenced. In this context, “differs from” refers to a difference of at least one amino acid relative to the homologous polypeptides encoded by the roan or sable antelopes. A non-coding or regulatory nucleic acid sequence can be considered a “bluebuck sequence” if a non-coding motif of at least 20 nucleotides is present in every bluebuck genome sequenced, and not present in any roan or sable antelope genome sequenced, or if one or more nucleotides in the regulatory region of every bluebuck genome sequence differs from one or more nucleotides in the regulatory region of the roan or sable antelope genomes. A roan or sable antelope gene or sequence modified by human intervention to encode a bluebuck gene or gene variant sequence is a bluebuck gene or gene or gene variant as the term is used herein. Where a bluebuck gene or gene variant as referred to herein is only found encoded in a bluebuck genome, and where the bluebuck is extinct, a bluebuck gene or gene variant sequence is necessarily exogenous to a viable cell; that is, the bluebuck gene or gene variant sequence is “exogenous” whether the sequence is in the cell through introduction of a foreign sequence or through gene editing an endogenous sequence to encode the bluebuck gene or gene variant sequence.

As identified herein, the bluebuck variants in the application were aligned to the roan antelope reference. The relevant gene sequences of the roan antelope reference are provided herein. An example of an additional roan antelope reference for alignment can include the GCA_016433095.1 roan antelope reference. This genome was assembled using shotgun sequencing, an older technology. The sequences provided herein were generated using current techniques, such as long-read genome sequencing. Production of a new assembly will lead to a change in the genomic coordinates of the variants listed in this application, as is standard for any species. To convert the coordinates listed here to any new assembly, a “liftover” can be conducted in accordance with standard procedures established by the genomics field.

Thus, provided herein are isolated nucleic acid sequences comprising one or more bluebuck (Hippotragus leucophaeus) gene variants associated with gray/blue coat color. The isolated nucleic acid sequences can, for example comprise one or more bluebuck gene variants associated with gray/blue coat color selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant.

Also provided are isolated nucleic acid sequences comprising one or more bluebuck (Hippotragus leucophaeus) gene variants associated with melanogenesis selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant. As used herein, the term “melanogenesis” refers to the production of melanin, which can pigment hair/fur and skin. Variants in melanogenesis genes can alter how and where melanin is produced in the body, as well as balance between pheomelanin (brown) and eumelanin (black).

Also provided are isolated nucleic acid sequences comprising one or more bluebuck (Hippotragus leucophaeus) gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the bluebuck gene variant comprises at least one change in the nucleotide sequence of the gene. The change in the nucleotide sequence can, for example, be a substitution, an insertion, a deletion, or a combination thereof. The substitution, the insertion, the deletion, or a combination thereof can, for example, be in a 5′ untranslated region of the gene, an intron of the gene, an exon of the gene, a 3′ untranslated region of the gene, or a combination thereof. The substitution, the insertion, the deletion, or a combination thereof can, for example, be in a regulatory region of the gene.

In certain embodiments, a bluebuck gene variant can comprise one or more substitutions within the regulatory region and/or within the coding region of the gene as compared to the corresponding reference genome. Thus, the bluebuck gene variant can comprise at least 1, at least 5, at least 10, at least 20, at least 50, at least 100, at least 150, at least 200, at least 250, at least 300, at least 350, at least 400, at least 450, at least 500 substitutions, at least 600 substitutions, or at least 700 substitutions within the regulatory region or within the coding region of the gene. The bluebuck variant can comprise about 1 to about 700, about 25 to about 675, about 50 to about 650, about 75 to about 625, about 100 to about 600, about 150 to about 550, about 200 to about 500 substitutions, and any value in between.

In certain embodiments, the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof. The bluebuck gene variant associated with gray/blue coat color can, for example, comprise a nucleotide sequence having 80%, 81%, 82%, 83%, 84%, 85%, 86%, 87%, 88%, 89%, 90%, 91%, 92%, 93%, 94%, 95%, 96%, 97%, 98%, or 99% identity to a nucleotide sequence selected from the group consisting of SEQ ID NOs: 45-102 and combinations thereof. The bluebuck gene variant associated with gray/blue coat color can, for example, comprise a nucleotide sequence selected from the group consisting of SEQ ID NOs: 147-204 and combinations thereof.

In certain embodiments, the bluebuck gene variant associated with melanogenesis comprises a nucleotide sequence having at least 80%, at least 85%, at least 90%, or at least 95% identity to a nucleotide sequence selected from the group consisting of 48, 64, 68-72, 120-146, and combinations thereof. The bluebuck gene variant associated with melanogenesis can, for example, comprise a nucleotide sequence having 80%, 81%, 82%, 83%, 84%, 85%, 86%, 87%, 88%, 89%, 90%, 91%, 92%, 93%, 94%, 95%, 96%, 97%, 98%, or 99% identity to a nucleotide sequence selected from the group consisting of SEQ ID NOs: 48, 64, 68-72, 120-146, and combinations thereof. The bluebuck gene variant associated with melanogenesis can, for example, comprise a nucleotide sequence selected from the group consisting of SEQ ID NOs: 150, 166, 170-174, 222-248, and combinations thereof.

In certain embodiments, the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof. The bluebuck gene variant associated with altered facial pattern can, for example, comprise a nucleotide sequence having 80%, 81%, 82%, 83%, 84%, 85%, 86%, 87%, 88%, 89%, 90%, 91%, 92%, 93%, 94%, 95%, 96%, 97%, 98%, or 99% identity to a nucleotide sequence selected from the group consisting of SEQ ID NOs: 60, 74, 76, 95, 103-119, and combinations thereof. The bluebuck gene variant associated with altered facial pattern can, for example, comprise a nucleotide sequence selected from the group consisting of SEQ ID NOs: 162, 176, 178, 197, 205-221, and combinations thereof.

In certain embodiments, the isolated nucleic acids comprise at least 2, 3, 4, 5, 6, 7, 8, 9, 10, 15, 20, 25, 30, 40, 50, 60, 70, 80, 90, 95, 100, 101, 102 (or any number in between) blue buck gene variants.

The exact location and substitution of a bluebuck gene variant can, for example, be determined by performing an alignment with the bluebuck sequence and roan antelope sequence provided herein in Table 1. The bluebuck gene variant can comprise at least one substitution, or more than one substitution, as compared to the roan antelope sequence. Thus, by way of an example, a bluebuck gene variant for ASIP can, for example, comprise 1, 5, 10, 15, 20 (or any number in between) substitutions in the gene, including in the intron, exon, or regulatory regions of the gene when compared to the roan antelope sequence. By way of another example, a bluebuck gene variant for MLPH can, for example, comprise 1, 2, 3, 4, 5, 6, 7, 8, 9, 10, 15, 20, 25, 30, 35, 40, 45, 50, 55, 60, 65, 70, 75, 76, 77, 78 (or any number in between) substitutions in the gene, including in the intron, exon, or regulatory regions of the gene when compared to the roan antelope sequence. A person skilled in the art will understand how to identify the substitutions and total number of substitutions using the bluebuck gene variant sequences and roan antelope sequences provided in Table 1.

TABLE 1
Bluebuck Gene Variants and Associated Phenotypes

| | | | | Roan | |
|---|---|---|---|---|---|
| | | | Total | Antelope | Bluebuck |
| | | | Variants | SEQ ID | SEQ ID |
| Gene Abb. | Gene Name | Phenotype | in Gene | NO | NO |
| ABCB6 | ATP Binding Cassette Subfamily | coat color | 4 | 45 | 147 |
| | B Member 6 (LAN Blood Group) | | | | |
| ACE2 | Angiotensin Converting Enzyme | coat color | 20 | 46 | 148 |
| | 2 | | | | |
| AP3B1 | Adaptor Related Protein | coat color | 267 | 47 | 149 |
| | Complex 3 Subunit Beta 1 | | | | |
| AP3D1 | Adaptor Related Protein | coat color/ | 39 | 48 | 150 |
| | Complex 3 Subunit Delta 1 | melanogenesis | | | |
| ASIP | Agouti Signaling Protein | coat color | 20 | 49 | 151 |
| ATP7A | ATPase Copper Transporting | coat color | 34 | 50 | 152 |
| | Alpha | | | | |
| BCL2 | BCL2 Apoptosis Regulator | coat color | 287 | 51 | 153 |
| BLOC1S2 | Biogenesis Of Lysosomal | coat color | 17 | 52 | 154 |
| | Organelles Complex 1 Subunit 2 | | | | |
| BLOC1S3 | Biogenesis Of Lysosomal | coat color | 4 | 53 | 155 |
| | Organelles Complex 1 Subunit 3 | | | | |
| BLOC1S4 | Biogenesis Of Lysosomal | coat color | 2 | 54 | 156 |
| | Organelles Complex 1 Subunit 4 | | | | |
| BLOC1S5 | Biogenesis Of Lysosomal | coat color | 51 | 55 | 157 |
| | Organelles Complex 1 Subunit 5 | | | | |
| BRAF | B-Raf Proto-Oncogene, | coat color | 196 | 56 | 158 |
| | Serine/Threonine Kinase | | | | |
| DCT | Dopachrome Tautomerase | coat color | 62 | 57 | 159 |
| DCTN1 | Dynactin Subunit 1 | coat color | 72 | 58 | 160 |
| DCTN2 | Dynactin Subunit 2 | coat color | 12 | 59 | 161 |
| DOCK7 | Dedicator Of Cytokinesis 7 | coat color/face | 238 | 60 | 162 |
| | | patterning | | | |
| DSTYK | Dual Serine/Threonine And | coat color | 73 | 61 | 163 |
| | Tyrosine Protein Kinase | | | | |
| DTNBP1 | Dystrobrevin Binding Protein 1 | coat color | 133 | 62 | 164 |
| EED | Embryonic Ectoderm | coat color | 38 | 63 | 165 |
| | Development | | | | |
| FIG4 | FIG4 Phosphoinositide 5- | coat color/ | 249 | 64 | 166 |
| | Phosphatase | melanogenesis | | | |
| GGT1 | Gamma-Glutamyltransferase 1 | coat color | 14 | 65 | 167 |
| GPR143 | G Protein-Coupled Receptor 143 | coat color | 37 | 66 | 168 |
| HPS1 | HPS1 Biogenesis Of Lysosomal | coat color | 91 | 67 | 169 |
| | Organelles Complex 3 Subunit 1 | | | | |
| HPS3 | HPS3 Biogenesis Of Lysosomal | coat color/ | 94 | 68 | 170 |
| | Organelles Complex 2 Subunit 1 | melanogenesis | | | |
| HPS4 | HPS4 Biogenesis Of Lysosomal | coat color/ | 27 | 69 | 171 |
| | Organelles Complex 3 Subunit 2 | melanogenesis | | | |
| HPS5 | HPS5 Biogenesis Of Lysosomal | coat color/ | 66 | 70 | 172 |
| | Organelles Complex 2 Subunit 2 | melanogenesis | | | |
| HPS6 | HPS6 Biogenesis Of Lysosomal | coat color/ | 1 | 71 | 173 |
| | Organelles Complex 2 Subunit 3 | melanogenesis | | | |
| LYST | Lysosomal Trafficking Regulator | coat color/ | 157 | 72 | 174 |
| | | melanogenesis | | | |
| MBTPS1 | Membrane Bound Transcription | coat color | 51 | 73 | 175 |
| | Factor Peptidase, Site 1 | | | | |
| MCOLN3 | Mucolipin TRP Cation Channel 3 | coat color/face | 61 | 74 | 176 |
| | | patterning | | | |
| MFSD12 | Major Facilitator Superfamily | coat color | 10 | 75 | 177 |
| | Domain Containing 12 | | | | |
| MITF | Melanocyte Inducing | coat color/face | 319 | 76 | 178 |
| | Transcription Factor | patterning | | | |
| MKLN1 | Muskelin 1 | coat color | 161 | 77 | 179 |
| MLANA | Melan-A | coat color | 8 | 78 | 180 |
| MLPH | Melanophilin | coat color | 78 | 79 | 181 |
| MREG | Melanoregulin | coat color | 80 | 80 | 182 |
| MYO5A | Myosin VA | coat color | 278 | 81 | 183 |
| NOTCH1 | Notch Receptor 1 | coat color | 31 | 82 | 184 |
| NOTCH2 | Notch Receptor 2 | coat color | 226 | 83 | 185 |
| OCA2 | OCA2 Melanosomal | coat color | 285 | 84 | 186 |
| | Transmembrane Protein | | | | |
| PAH | Phenylalanine Hydroxylase | coat color | 125 | 85 | 187 |
| PIKFYVE | Phosphoinositide Kinase, FYVE- | coat color | 107 | 86 | 188 |
| | Type Zinc Finger Containing | | | | |
| PMEL | Premelanosome Protein | coat color | 7 | 87 | 189 |
| RAB27A | RAB27A, Member RAS | coat color | 123 | 88 | 190 |
| | Oncogene Family | | | | |
| RAB38 | RAB38, Member RAS Oncogene | coat color | 112 | 89 | 191 |
| | Family | | | | |
| RABGGTA | Rab Geranylgeranyltransferase | coat color | 7 | 90 | 192 |
| | Subunit Alpha | | | | |
| RAF1 | Raf-1 Proto-Oncogene, | coat color | 32 | 91 | 193 |
| | Serine/Threonine Kinase | | | | |
| SLC24A5 | Solute Carrier Family 24 Member | coat color | 100 | 92 | 194 |
| | 5 | | | | |
| SLC45A2 | Solute Carrier Family 45 Member | coat color | 42 | 93 | 195 |
| | 2 | | | | |
| SLC7A11 | Solute Carrier Family 7 Member | coat color | 122 | 94 | 196 |
| | 11 | | | | |
| SNAI2 | Snail Family Transcriptional | coat color/face | 2 | 95 | 197 |
| | Repressor 2 | patterning | | | |
| SPAG9 | Sperm Associated Antigen 9 | coat color | 118 | 96 | 198 |
| TPCN2 | Two Pore Segment Channel 2 | coat color | 18 | 97 | 199 |
| TYR | Tyrosinase | coat color | 145 | 98 | 200 |
| TYRP1 | Tyrosinase Related Protein 1 | coat color | 20 | 99 | 201 |
| VAC14 | VAC14 Component Of | coat color | 97 | 100 | 202 |
| | PIKFYVE Complex | | | | |
| VPS33A | VPS33A Core Subunit Of | coat color | 35 | 101 | 203 |
| | CORVET And HOPS Complexes | | | | |
| YKT6 | YKT6 V-SNARE Homolog | coat color | 14 | 102 | 204 |
| ADAMTS20 | ADAM Metallopeptidase With | face patterning | 300 | 103 | 205 |
| | Thrombospondin Type 1 Motif | | | | |
| | 20 | | | | |
| BMP4 | Bone Morphogenetic Protein 4 | face patterning | 4 | 104 | 206 |
| EDN3 | Endothelin 3 | face patterning | 35 | 105 | 207 |
| EDNRB | Endothelin Receptor Type B | face patterning | 37 | 106 | 208 |
| ETS1L1 | ETS Proto-Oncogene 1, | face patterning | 112 | 107 | 209 |
| | Transcription Factor Like 1 | | | | |
| ETS1L2 | ETS Proto-Oncogen 1, | face patterning | 47 | 108 | 210 |
| | Transcription Factor Like 2 | | | | |
| FOXD3 | Forkhead Box D3 | face patterning | 1 | 109 | 211 |
| HDAC1 | Histone Deacetylase 1 | face patterning | 32 | 110 | 212 |
| IRF1 | Interferon Regulatory Factor 1 | face patterning | 10 | 111 | 213 |
| KIT | KIT Proto-Oncogene, Receptor | face patterning | 95 | 112 | 214 |
| | Tyrosine Kinase | | | | |
| KITLG | KIT Ligand | face patterning | 111 | 113 | 215 |
| PAX3 | Paired Box 3 | face patterning | 150 | 114 | 216 |
| SOX10 | SRY-Box Transcription Factor | face patterning | 14 | 115 | 217 |
| | 10 | | | | |
| SOX2 | SRY-Box Transcription Factor 2 | face patterning | 21 | 116 | 218 |
| TRPM1 | Transient Receptor Potential | face patterning | 76 | 117 | 219 |
| | Cation Channel Subfamily M | | | | |
| | Member 1 | | | | |
| WNT3A | Wnt Family Member 3A | face patterning | 91 | 118 | 220 |
| ZFHX4 | Zinc Finger Homeobox 4 | face patterning | 182 | 119 | 221 |
| ASCC2 | Activating Signal Cointegrator 1 | melanogenesis | 62 | 120 | 222 |
| | Complex Subunit 2 | | | | |
| ATP6V0A2 | ATPase H+ Transporting V0 | melanogenesis | 60 | 121 | 223 |
| | Subunit A2 | | | | |
| CCDC53 | NA | melanogenesis | 44 | 122 | 224 |
| CD72 | CD72 Molecule | melanogenesis | 2 | 123 | 225 |
| CDK12 | Cyclin Dependent Kinase 12 | melanogenesis | 39 | 124 | 226 |
| CHD8 | Chromodomain Helicase DNA | melanogenesis | 42 | 125 | 227 |
| | Binding Protein 8 | | | | |
| COMMD5 | COMM Domain Containing 5 | melanogenesis | 2 | 126 | 228 |
| DSCR3 | NA | melanogenesis | 33 | 127 | 229 |
| EP300 | E1A Binding Protein P300 | melanogenesis | 56 | 128 | 230 |
| FRYL | FRY Like Transcription | melanogenesis | 267 | 129 | 231 |
| | Coactivator | | | | |
| HDAC4 | Histone Deacetylase 4 | melanogenesis | 270 | 130 | 232 |
| HNRNPA2B1L1 | Heterogeneous Nuclear | melanogenesis | 24 | 131 | 233 |
| | Ribonucleoprotein A2/B1 Like 1 | | | | |
| HNRNPA2B1L2 | Heterogeneous Nuclear | melanogenesis | 15 | 132 | 234 |
| | Ribonucleoprotein A2/B1 Like 2 | | | | |
| HNRNPA2B1L3 | Heterogeneous Nuclear | melanogenesis | 2 | 133 | 235 |
| | Ribonucleoprotein A2/B1 Like 3 | | | | |
| HNRNPA2B1L4 | Heterogeneous Nuclear | melanogenesis | 1 | 134 | 236 |
| | Ribonucleoprotein A2/B1 Like 4 | | | | |
| INTS8 | Integrator Complex Subunit 8 | melanogenesis | 58 | 135 | 237 |
| NCOR1 | Nuclear Receptor Corepressor 1 | melanogenesis | 100 | 136 | 238 |
| RAB9A | RAB9A, Member RAS | melanogenesis | 15 | 137 | 239 |
| | Oncogene Family | | | | |
| RB1 | RB Transcriptional Corepressor 1 | melanogenesis | 169 | 138 | 240 |
| SCAF4 | SR-Related CTD Associated | melanogenesis | 81 | 139 | 241 |
| | Factor 4 | | | | |
| SEC31A | SEC31 Homolog A, COPII Coat | melanogenesis | 84 | 140 | 242 |
| | Complex Component | | | | |
| SIAH1 | Siah E3 Ubiquitin Protein Ligase | melanogenesis | 23 | 141 | 243 |
| | 1 | | | | |
| SMG7 | SMG7 Nonsense Mediated | melanogenesis | 81 | 142 | 244 |
| | MRNA Decay Factor | | | | |
| SNX13L1 | Sorting Nexin 13 Like 1 | melanogenesis | 139 | 143 | 245 |
| SNX13L2 | Sorting Nexin 13 Like 2 | melanogenesis | 1 | 144 | 246 |
| TBC1D5 | TBC1 Domain Family Member 5 | melanogenesis | 650 | 145 | 247 |
| WDFY1 | WD Repeat And FYVE Domain | melanogenesis | 74 | 146 | 248 |
| | Containing 1 | | | | |

Total Variants in Gene can include variants in introns, exons, and regulatory regions.

Also provided are isolated vectors comprising the isolated nucleic acid sequences described herein.

Also provided are recombinant host cells comprising one or more bluebuck (Hippotragus leucophaeus) gene variants associated with gray/blue coat color and/or melanogenesis. In certain embodiments, the recombinant host cells comprise one or more bluebuck gene variants one or more bluebuck gene variants associated with gray/blue coat color selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant. In certain embodiments, the recombinant host cells comprise one or more bluebuck gene variants associated with melanogenesis selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant.

Also provided are recombinant host cells comprising one or more bluebuck (Hippotragus leucophaeus) gene variants associated with altered facial pattern. In certain embodiments, the recombinant host cells comprise one or more bluebuck gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Also provided are recombinant host cells comprising one or more bluebuck (Hippotragus leucophaeus) gene variants associated with gray/blue coat color. The recombinant host cells comprise one or more bluebuck gene variants are selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant.

In certain embodiments, the recombinant host cell comprises 2, 3, 4, 5, 6, 7, 8, 9, 10, 15, 20, 25, 30, 40, 50, 60, 70, 80, 90, 95, 100, 101, 102 (or any number in between) bluebuck gene variants. In some embodiments, the bluebuck gene variant comprises one or more substitutions within the regulatory region and/or within the coding region of the gene as compared to a corresponding reference genome, wherein the reference genome is the genome of the host cell prior to the one or more substitutions used to generate the recombinant cell.

In certain embodiments, in recombinant host cells comprising one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the recombinant host cell can further comprise a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, in recombinant host cells comprising one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the recombinant host cell can further comprise a bluebuck gene variant associated with gray/blue coat color selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant. In certain embodiments, (a) the loss-of-function ASIP variant comprises an insertion and deletion (indel); (b) the DEFB103 missense variant comprises a glycine deletion at an amino acid position corresponding to position 23 of SEQ ID NO:4; (c) the loss-of-function MLPH variant comprises an indel; (d) the PMEL missense variant comprises a cysteine substitution for an arginine at an amino acid position corresponding to position 603 of SEQ ID NO: 8; (e) the loss-of-function LYST variant comprises an indel; and/or (f) the loss-of-function SLC45A2 variant comprises an indel. In certain embodiments, the recombinant host cell can further comprise a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

As used herein, insertions and deletions (indels) can be used to create loss-of-function variants of the targeted protein. The loss-of-function variants can either be transcribed at the RNA level and degraded by a process known as nonsense mediated decay (NMD). Alternatively, the loss-of-function variant can be translated into a shortened peptide without the functional domains of the full-length protein. A person skilled in the art would understand how to create an insertion or deletion in a peptide to arrive at a variant with a loss-of-function.

As used herein, missense variants are single substitutions of one amino acid for another amino acid resulting in a missense variant. A person skilled in the art would understand how to create a missense variant.

In certain embodiments, the recombinant host cell fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the recombinant host cell fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

Also provided are recombinant host cells comprising at least one of the isolated nucleic acid sequences described herein. In certain embodiments, the recombinant host cell comprises 1, 2, 3, 4, 5, 6, 7, 8, 9, 10, 15, 20, 25, 30, 40, 50, 60, 70, 80, 90, 95, 100, 101, 102 (or any number in between) of the isolated nucleic acids described herein.

Also provided herein are recombinant host cells comprising a deletion of at least one endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the recombinant host cell comprises a deletion of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the recombinant host cells comprising the isolated nucleic acids described herein further comprise a deletion of at least one endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the recombinant host cell is an antelope cell. In certain embodiments, the recombinant host cell is a roan antelope (Hippotragus equinus) cell or a sable antelope (Hippotragus niger) cell.

The bluebuck gene variants described herein can be used in any combination to be expressed in any recombinant host cell as described herein.

### Cells

The bluebuck gene variants described herein can be expressed by any viable cell that can accept exogenous genetic material. The cell can be, for example, a prokaryotic cell or a eukaryotic cell. In some embodiments, the cell is a eukaryotic cell. The cell can be a reprogrammed cell, a non-human oocyte, a cell of a non-human embryo or a cell of a non-human blastula. In some embodiments of any of the aspects, the cell is a fibroblast cell. In some embodiments, the cell is selected from the group consisting of a nerve cell, a cartilage cell, a bone cell, a muscle cell, a bone cell, a fat cell, and an epidermal cell. In some embodiments, the cell was previously differentiated into a cell selected from the group consisting of a nerve cell, cartilage cell, bone cell, muscle cell, bone cell, fat cell, and an epidermal cell.

The scientific literature provides guidance for one of ordinary skill in the art to isolate and prepare cells as necessary for use with the isolated nucleic acids and vectors described herein.

The cells described herein can be from any viable non-human source or organism. Usually, the organism is an animal or vertebrate such as a wild animal, zoo animal, endangered animal, rodent, domestic animal, or bird. Animals can include, as non-limiting examples, an antelope, hippopotamus, hyrax, manatee, bear, panda, feline species, e.g., tiger, lion, cheetah, bobcat, canine species, e.g., fox, wolf, avian species, e.g., ostrich, emu, penguin, pigeon, and fish, e.g., trout, catfish, and salmon. In some embodiments, the cell described herein is from a mammal. A non-limiting example of an organism from which cells can be derived includes antelopes (e.g., Hippotragus equinus and Hippotragus niger).

In certain embodiments, a cell useful in the methods and compositions described herein is an antelope cell. In some embodiments, the cell is an antelope fibroblast cell. In some embodiments, the cell is an antelope stem cell. In some embodiments, the cell described herein is an antelope somatic cell reprogrammed to a stem cell or stem cell-like phenotype having stem cell-like morphology and/or expressing at least one stem cell marker described herein.

The cells described herein can be from any tissue isolated from an organism by methods known in the art. For example, placental tissue can be isolated from a given organism (e.g., an antelope), after full term delivery of young, and subsequently processed for cellular isolation and/or culture by methods known in the art. Additional exemplary cell types that can be used for the compositions and methods described herein include but are not limited to fibroblasts, skin cells, blood cells (e.g., leukocytes, monocytes, dendritic cells), stem cells, hematopoietic cells, liver cells, vascular cells, muscle cells, pancreatic cells, neural cells, ocular or retinal cells, epithelial or endothelial cells, lung cells, cardiac cells, intestinal cells, diaphragmatic cells, renal (i.e., kidney) cells, bone marrow cells, or any one or more selected tissues or cells of an organism for which genetic modification or gene editing to express a bluebuck gene is contemplated.

In certain embodiments, the isolated nucleic acids and vectors described herein are used in stem cells. Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include, for example, amnion-derived or placental-derived stem cells. Pluripotent stem cells can differentiate into cells derived from any of the three germ layers.

In certain embodiments, the recombinant host cell is a stem cell. The stem cell can, for example, be selected from an induced stem cell, embryonic stem (ES) cell, or a mesenchymal stem cell (MSC). In certain embodiments, the recombinant host cell is a reprogrammed cell. In certain embodiments, the recombinant host cell is a fibroblast cell or a mesenchymal cell. In certain embodiments, the recombinant host cell is selected from the group consisting of a nerve cell, a cartilage cell, a bone cell, a muscle cell, a bone cell, a fat cell, and an epidermal cell.

In certain embodiments, the recombinant host cell fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RBI, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the recombinant host cell fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the recombinant host cell is an antelope cell. In certain embodiments, the recombinant host cell is a roan antelope (Hippotragus equinus) cell or a sable antelope (Hippotragus niger) cell.

Also provided are transgenic animals comprising a recombinant host cell as described herein. In certain embodiments, the transgenic animal is an antelope. In certain embodiments, the transgenic animal is a roan antelope (Hippotragus equinus) or a sable antelope (Hippotragus niger).

### Transgenic Animals

Provided herein are transgenic animals comprising one or more bluebuck (Hippotragus leucophaeus) gene variants associated with gray/blue coat color and/or melanogenesis. The transgenic animals can, for example comprise one or more bluebuck gene variants associated with gray/blue coat color selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant. The transgenic animals can, for example comprise one or more bluebuck gene variants associated with melanogenesis selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RBI variant, a SCAF4 variant, a SEC31A variant, a SIAH1 variant, a SMG7 variant, a SNX13L1 variant, a SLX13L2 variant, a TBC1D5 variant, and a WDFY1 variant.

Also provided are transgenic animals comprising one or more bluebuck (Hippotragus leucophaeus) gene variants associated with altered facial pattern. The transgenic animal can, for example, comprise one or more bluebuck gene variants associated with altered facial pattern are selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Also provided are transgenic animals comprising one or more bluebuck (Hippotragus leucophaeus) gene variants associated with gray/blue coat color. The one or more bluebuck gene variants can, for example, be selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant.

In certain embodiments, the transgenic animal comprises 2, 3, 4, 5, 6, 7, 8, 9, 10, 15, 20, 25, 30, 40, 50, 60, 70, 80, 90, 95, 100, 101, 102 (or any number in between) bluebuck gene variants. In some embodiments, the bluebuck gene variant comprises one or more substitutions within the regulatory region and/or within the coding region of the gene as compared to a corresponding reference genome, wherein the corresponding reference genome is the genome of the animal prior to the one or more substitutions.

In certain embodiments, the ASIP variant, the LYST variant, the SLC45A2 variant, the BCL2 variant, the DCT variant, and/or the MYO5A variant is in a coding region. In certain embodiments, (a) the ASIP variant in the coding region is selected from an isoleucine substitution for valine at amino acid position 39; and/or a tryptophan substitution for arginine at amino acid position 10, wherein the amino acid position corresponds to SEQ ID NO:2; (b) the LYST variant in the coding region is selected from a tyrosine substitution for a cysteine at amino acid position 1352, and/or an arginine substitution for a valine at amino acid position 1079, wherein the amino acid position corresponds to SEQ ID NO:10; (c) the SLC45A2 variant in the coding region is selected from a valine substitution for a methionine at amino acid position 475, and/or a cysteine substitution for an arginine at amino acid position 59, wherein the amino acid position corresponds to SEQ ID NO:12; (d) the BCL2 variant in the coding region is selected from a threonine substitution for an asparagine at amino acid position 139, and/or an aspartic acid substitution for a glycine at amino acid position 127, wherein the amino acid position corresponds to SEQ ID NO: 14; (e) the DCT variant in the coding region is selected from a phenylalanine substitution for a leucine at amino acid position 240, and/or an arginine substitution for a tryptophan at amino acid position 22, wherein the amino acid position corresponds to SEQ ID NO: 16; and/or (f) the MYO5A variant in the coding region is selected from a serine substitution for a proline at amino acid position 1321, and/or a serine substitution for an asparagine at amino acid position 372, wherein the amino acid position corresponds to SEQ ID NO:20.

In certain embodiments, the ASIP variant, the MLPH variant, and/or the LYST variant is in a regulatory region. In certain embodiments, (a) the ASIP variant in the regulatory region is selected from an adenine substitution for a guanine at nucleotide position 919, a thymine substitution for a guanine at nucleotide position 1530, a thymine substitution for an adenine at nucleotide position 1578, a thymine substitution for a cytosine at nucleotide position 3703, an adenine substitution for a guanine at nucleotide position 3892, a cytosine substitution for a thymine at nucleotide position 4274, a thymine substitution for an adenine at nucleotide position 4370, a thymine substitution for a cytosine at nucleotide position 4807, a guanine substitution for an adenine at nucleotide position 4821, and/or an adenine substitution for a guanine at nucleotide position 4905, wherein the nucleotide position corresponds to SEQ ID NO:25; (b) the MLPH variant in the regulatory region is selected from a cytosine substitution for a guanine at nucleotide position 1238, an adenine substitution for a thymine at nucleotide position 1299; an adenine substitution for a cytosine at nucleotide position 1488, an adenine substitution for a guanine at nucleotide position 1526, a guanine substitution for a thymine at nucleotide position 1618, an adenine substitution for a guanine at nucleotide position 1653, an adenine substitution for a guanine at nucleotide position 1672; a thymine substitution for a cytosine at nucleotide position 1718, an adenine substitution for a guanine at nucleotide position 1837, a guanine substitution for an adenine at nucleotide position 1949, an adenine substitution for a cytosine at nucleotide position 2472, a guanine substitution for an adenine at nucleotide position 2675, a thymine substitution for a cytosine at nucleotide position 2691, a guanine substitution for a thymine at nucleotide position 2900, a guanine substitution for an adenine at nucleotide position 3029, a guanine substitution for an adenine at nucleotide position 3090, a guanine substitution for a thymine at nucleotide position 3272, a cytosine substitution for a thymine at nucleotide position 3380, a cytosine substitution for a thymine at nucleotide position 3449, a cytosine substitution for a thymine at nucleotide position 3463, an adenine substitution for a guanine at nucleotide position 3513, a cytosine substitution for a thymine at nucleotide position 3559, a cytosine substitution for a thymine at nucleotide position 3581, a guanine substitution for a cytosine at nucleotide position 4352, a guanine substitution for a cytosine at nucleotide position 4356, a guanine substitution for an adenine at nucleotide position 4552, a guanine substitution for an adenine at nucleotide position 4690, a guanine substitution for an adenine at nucleotide position 4856, a cytosine substitution for a thymine at nucleotide position 4888, and/or a thymine substitution for a cytosine at nucleotide position 4990, wherein the nucleotide position corresponds to SEQ ID NO:27; and/or (c) the LYST variant in the regulatory region is selected from a cytosine substitution for a guanine at nucleotide position 48, a cytosine substitution for an adenine at nucleotide position 67, a guanine substitution for a cytosine at nucleotide position 71, a thymine substitution for a cytosine at nucleotide position 247, a cytosine substitution for a thymine at nucleotide position 254, a guanine substitution for an adenine at nucleotide position 486, a thymine substitution for a cytosine at nucleotide position 883, a cytosine substitution for an adenine at nucleotide position 1002, an adenine substitution for a guanine at nucleotide position 1105, a thymine substitution for a cytosine at nucleotide position 1505, an adenine substitution for a guanine at nucleotide position 2350, a thymine substitution for a cytosine at nucleotide position 2612, a thymine substitution for a cytosine at nucleotide position 2661, a cytosine substitution for a thymine at nucleotide position 2668, a cytosine substitution for a thymine at nucleotide position 2818, a cytosine substitution for a guanine at nucleotide position 2873, a cytosine substitution for a guanine at nucleotide position 3223, an adenine substitution for a guanine at nucleotide position 3257, a thymine substitution for a cytosine at nucleotide position 4347, a cytosine substitution for a thymine at nucleotide position 4420, a thymine substitution for a cytosine at nucleotide position 4586, a cytosine substitution for a thymine at nucleotide position 4598, and/or a thymine substitution for a cytosine at nucleotide position 4619, wherein the nucleotide position corresponds to SEQ ID NO:29.

In certain embodiments, wherein the transgenic animals comprises one or more bluebuck gene variants associated with gray/blue coat color selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant, the transgenic animal can further comprise one or more bluebuck gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the MITF variant and the KIT variant is in a regulatory region. In certain embodiments, (a) the MITF variant in the regulatory region is selected from a thymine substitution for a cytosine at nucleotide position 514, an adenine substitution for a guanine at nucleotide position 6733, a thymine substitution for an adenine at nucleotide position 21322, an adenine substitution for a thymine at nucleotide position 29009, a thymine substitution for a cytosine at nucleotide position 37136, an adenine substitution for a thymine adenine at nucleotide position 48433, a thymine substitution for a guanine at nucleotide position 50738, a thymine substitution for a guanine at nucleotide position 52771, a thymine substitution for an adenine at nucleotide position 53986, a guanine substitution for an adenine at nucleotide position 85470, an adenine substitution for a guanine at nucleotide position 85945, a guanine substitution for a thymine at nucleotide position 89390, an adenine substitution for a guanine at nucleotide position 91717, a cytosine substitution for an adenine at nucleotide position 110490, an adenine substitution for a guanine at nucleotide position 114903, an adenine substitution for a guanine at nucleotide position 116734, an adenine substitution for a guanine at nucleotide position 119238, a cytosine substitution for an adenine at nucleotide position 122369, an adenine substitution for a cytosine at nucleotide position 126635, an adenine substitution for a guanine at nucleotide position 129446, a thymine substitution for a guanine at nucleotide position 133318, an adenine substitution for a guanine at nucleotide position 138944, a cytosine substitution for a thymine at nucleotide position 150167, an adenine substitution for a cytosine at nucleotide position 187625, a guanine substitution for an adenine at nucleotide position 208940, an adenine substitution for a guanine at nucleotide position 211614, a guanine substitution for an adenine at nucleotide position 216326, and/or a cytosine substitution for a thymine at nucleotide position 217379, wherein the nucleotide position corresponds to SEQ ID NO:33; and/or (b) the KIT variant in the regulatory region is selected from a thymine substitution for a cytosine at nucleotide position 268, a cytosine substitution for a guanine at nucleotide position 510, a guanine substitution for an adenine at nucleotide position 517, an adenine substitution for a guanine at nucleotide position 2108, a thymine substitution for a cytosine at nucleotide position 2401, an adenine substitution for a thymine at nucleotide position 2504, a cytosine substitution for a thymine at nucleotide position 2835, a guanine substitution for a adenine at nucleotide position 3365, a cytosine substitution for a thymine at nucleotide position 4044, an adenine substitution for a guanine at nucleotide position 4166, an adenine substitution for a cytosine at nucleotide position 4531, an adenine substitution for a guanine at nucleotide position 4569, a thymine substitution for a guanine at nucleotide position 4714, an adenine substitution for a cytosine at nucleotide position 4859, an adenine substitution for a guanine at nucleotide position 4892, and/or a guanine substitution for an adenine at nucleotide position 4907, wherein the nucleotide position corresponds to SEQ ID NO:31.

In certain embodiments, wherein the transgenic animal comprises one or more bluebuck gene variants associated with gray/blue coat color and melanogenesis selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant, the transgenic animal can further comprise one or more bluebuck gene variants associated with gray/blue coat color selected from (a) a loss-of-function ASIP variant; (b) a DEFB103 missense variant; (c) a loss-of-function MLPH variant; (d) a PMEL missense variant; (e) a loss-of-function LYST variant; and/or (f) a loss-of-function SLC45A2 variant. In certain embodiments, (a) the loss-of-function ASIP variant comprises an insertion and deletion (indel); (b) the DEFB103 missense variant comprises a glycine deletion at an amino acid position corresponding to position 23 of SEQ ID NO:4; (c) the loss-of-function MLPH variant comprises an indel; (d) the PMEL missense variant comprises a cysteine substitution for an arginine at an amino acid position corresponding to position 603 of SEQ ID NO: 8; (e) the loss-of-function LYST variant comprises an indel; and (f) the loss-of-function SLC45A2 variant comprises an indel. In certain embodiments, the transgenic animal can further comprise one or more bluebuck gene variants associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

In certain embodiments, the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In certain embodiments, the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

In certain embodiments, the transgenic animal is an antelope. In certain embodiments, the transgenic animal is a roan antelope (Hippotragus equinus) or a sable antelope (Hippotragus niger).

Methods for Introducing Bluebuck Gene Variants or Deletions of Regulatory Elements into a Cell

In certain embodiments of any of the aspects, the cell compositions described herein express a polypeptide encoded by the at least one isolated nucleic acid sequence having a bluebuck gene variant nucleotide sequence.

The cells described herein can be transfected, contacted with, or administered an exogenous bluebuck gene encoded by the isolated nucleic acids described herein by methods known in the art.

In some embodiments, the at least one nucleic acid sequence encoding a bluebuck gene is delivered via a vector.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc.

In some embodiments of any of the aspects, the vector is selected from the group consisting of a plasmid, a cosmid, and a viral vector.

An expression vector is a vector that directs expression of an RNA or polypeptide (e.g., a bluebuck polypeptide) from nucleic acid sequences contained therein linked to transcriptional regulatory sequences on the vector. The sequences expressed will often, but not necessarily, be heterologous to the cell; a bluebuck gene introduced to a viable cell is heterologous to the cell. An expression vector may comprise additional elements, for example, the expression vector may have two replication systems, thus allowing it to be maintained in two organisms, for example in animal cells for expression and in a prokaryotic host for cloning and amplification. “Expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

In some embodiments, a vector is capable of driving expression of one or more sequences in a mammalian cell; i.e., the vector is a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

### Methods of Inhibiting or Editing the Expression of an Endogenous Gene

In some embodiments of any of the disclosed aspects, the cell described herein does not express an endogenous homologue of the at least one bluebuck gene variant described herein. In another embodiment of any of the aspects, the cell is edited to inhibit expression of an endogenous homologue of the at least one bluebuck gene variant. In another embodiment of any of the aspects, the cell is edited to alter the regulatory and/or coding region to incorporate substitutions so that the endogenous homologue resembles the at least one bluebuck gene variant.

In another embodiment of any of the aspects, the non-bluebuck homologue of the exogenous nucleic acid sequence has been deleted or inactivated.

It is contemplated herein that when one or more bluebuck gene variants are delivered to the host cell(s) it can be advantageous to modify the endogenous non-bluebuck homologue of the one or more genes to render the endogenous gene or genes non-functional. It is further contemplated herein that if two or more bluebuck genes are delivered to the host cell, one or both of the endogenous host cell genes would be altered. Thus, in this context, the host cell can comprise at least one non-functional endogenous homologue to the corresponding bluebuck gene.

In the context of antelope cells, the antelope homologue(s) of the one or more bluebuck genes to be expressed would be altered, deleted, or inhibited such that only the one or more bluebuck genes is/are expressed by the cell. This can be achieved, for example, by standard gene editing of target sequences. It is also contemplated that rather than simply inactivating the endogenous gene, wholesale replacement of the endogenous gene, e.g., via homologous recombination, or via selective editing of the non-bluebuck homologue gene(s) to encode and express the bluebuck variant gene sequence(s) could also be performed.

The target sequence can be determined by methods known in the art. For example, sequence alignment tools can be used to compare the bluebuck nucleic acid sequences to those in the host organism, e.g., using NCBI Basic Local Alignment Sequence Tool (BLAST), OrthoMaM, Ensembl and/or Megalign (DNASTAR) software. Those skilled in the art can determine appropriate parameters for aligning sequences, including any algorithms needed to achieve maximal alignment over the full length of the sequences being compared.

Methods of inhibiting gene function in a host cell are known in the art. Non-limiting examples of gene knockdown, inhibition, and alteration include, e.g., gene editing enzymes, Transcription Activator-Like Effectors Nucleases (TALENS), and inhibitory nucleic acids. Exemplary embodiments of types of inhibitory nucleic acids can include, e.g., siRNA, shRNA, miRNA, and/or a miRNA, which are known in the art. One of ordinary skill in the art can design and test an inhibitory agent that targets the endogenous homologue of a bluebuck gene variant described herein.

Methods of preparing and delivering gene editing systems are described, e.g., in WO2015/013583 A2; U.S. Pat. No. 10,640,789 B2; US Publication No. US2019/0367948 A1; US Publication No. 2017/0266320 A1; US Publication No. 2018/0171361 A1; US Publication No. 2016/017546 2 A1; and US Publication No. 2018/0195089 A1, the contents of each of which are incorporated herein by reference in their entirety.

### Methods of Generating Transgenic Animals

The disclosure also provides for methods of generating a transgenic animal with the gray/blue coat color associated with the bluebuck antelope (Hippotragus leucophaeus). In certain embodiments, the methods can be used to generate the transgenic animals described above.

One embodiment of the disclosure is directed to methods of creating a transgenic animal with gray/blue coat color and/or a transgenic animal with an altered melanin biosynthesis pathway of a bluebuck by introducing in a cell from a source animal (such as a roan antelope (Hippotragus equinus) or a sable antelope (Hippotragus niger)) at least one or more bluebuck (Hippotragus leucophaeus) gene variants associated with gray/blue coat color and/or melanogenesis and then utilizing the recombinant cell to produce a transgenic animal that has a gray/blue coat color and/or a transgenic animal with the melanogenesis of a bluebuck. The one or more bluebuck gene variants associated with gray/blue coat color and/or melanogenesis are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant.

In one embodiment, the method of creating a transgenic animal with gray/blue coat color and/or an altered melanin biosynthesis pathway of a bluebuck includes: introducing into the cell at least one or more bluebuck (Hippotragus leucophaeus) gene variants associated with gray/blue coat color and/or melanogenesis, wherein the one or more bluebuck gene variants are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant; and utilizing the recombinant cell to produce a transgenic animal that has a gray/blue coat color and/or an altered melanin biosynthesis pathway of a bluebuck.

In another embodiment, the method of creating a transgenic animal with gray/blue coat color and/or an altered melanin biosynthesis pathway of a bluebuck includes: obtaining a cell from the animal; introducing into the cell at least one bluebuck gene variant associated with gray/blue coat color and/or melanogenesis, wherein the one or more bluebuck gene variants are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant; and utilizing the recombinant cell to produce a transgenic animal, whereby the transgenic animal has a gray/blue coat color and/or an altered melanin biosynthesis pathway of a bluebuck.

The number of bluebuck gene variants introduced into the cell may vary. In certain embodiments, 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) bluebuck gene variants are introduced into the cell. In some embodiments, the bluebuck gene variant comprises one or more substitutions within the regulatory region and/or within the coding region of the gene as compared to a corresponding reference genome, wherein the corresponding reference genome is the genome of the cell prior to the one or more substitutions.

In some embodiments, the transgenic animal further also includes: a loss-of-function ASIP variant; a DEFB103 missense variant; a loss-of-function MLPH variant; a PMEL missense variant; a loss-of-function LYST variant; and/or a loss-of-function SLC45A2 variant. In other embodiments, the transgenic animal further includes a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Another embodiment of the disclosure is directed to methods of creating a transgenic animal with gray/blue coat color by introducing into a cell from a source animal (such as a roan antelope (Hippotragus equinus) or a sable antelope (Hippotragus niger)) at least one variant selected from: a loss-of-function ASIP variant; a DEFB103 missense variant; a loss-of-function MLPH variant; a PMEL missense variant; a loss-of-function LYST variant; and/or a loss-of-function SLC45A2 variant, and thereby produce a recombinant cell. The recombinant cell is then used to produce a transgenic animal having a gray/blue coat color.

In certain embodiments, the methods of creating a transgenic animal with gray/blue coat color include: introducing into an animal cell at least one bluebuck gene variant associated with gray/blue coat color selected from: a loss-of-function ASIP variant; a DEFB103 missense variant; a loss-of-function MLPH variant; a PMEL missense variant; a loss-of-function LYST variant; and/or a loss-of-function SLC45A2 variant, whereby introducing the at least one bluebuck gene variant produces a recombinant cell; and then utilizing the recombinant cell to produce a transgenic animal having a gray/blue coat color.

In other embodiments, the methods of creating a transgenic animal with gray/blue coat color include (a) obtaining a cell from the animal; (b) introducing into the cell at least one variant selected from: a loss-of-function ASIP variant; a DEFB103 missense variant; a loss-of-function MLPH variant; a PMEL missense variant; a loss-of-function LYST variant; and/or a loss-of-function SLC45A2 variant, whereby introducing the at least one variant produces a recombinant cell; and (c) utilizing the recombinant cell to produce a transgenic animal having a gray/blue coat color.

In certain embodiments, the transgenic animal further includes a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

Yet another embodiment of the disclosure is directed to methods of creating a transgenic animal with an altered facial pattern by introducing into cell from a source animal (such as a roan antelope (Hippotragus equinus) or a sable antelope (Hippotragus niger)) a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant. The recombinant cell having these features is then used to produce a transgenic animal having an altered facial pattern.

In some embodiments, the methods for creating a transgenic animal with an altered facial pattern include: introducing into an animal cell a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant; and utilizing the recombinant cell to produce a transgenic animal having an altered facial pattern.

In other embodiments, the methods for creating a transgenic animal with an altered facial pattern include: obtaining a cell from the animal; introducing into the cell a bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant; and utilizing the recombinant cell to produce a transgenic animal, whereby the transgenic animal has an altered facial pattern.

In certain embodiments of any of the methods of creating a transgenic animal, the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RBI, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4. In some embodiments, the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RBI, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

A variety of cells may be used in the methods of creating a transgenic animal. In one embodiment, the cell is a stem cell. In another embodiment, the cell is a stem cell selected from an induced stem cell, an embryonic stem (ES) cell, a mesenchymal stem cell (MSC), or combinations thereof. In an alternate embodiment, the cell is a reprogrammed cell. In yet another embodiment, the cell is a fibroblast cell or a mesenchymal cell. In a further embodiment, the cell is an endothelial progenitor cell (EPC) or a pericyte. In a further embodiment, the cell is selected from the group consisting of a nerve cell, a cartilage cell, a bone cell, a muscle cell, a fat cell, and an epidermal cell.

In certain embodiments of any of the methods of creating a transgenic animal, the animal is an antelope. In other embodiments, the antelope is selected from a roan antelope (Hippotragus equinus) or a sable antelope (Hippotragus niger).

The disclosure also includes a transgenic animal made by these methods.

### EMBODIMENTS

The invention provides also the following non-limiting embodiments.
Embodiment 1 is a transgenic animal comprising at least one bluebuck (Hippotragus leucophaeus) gene variant associated with gray/blue coat color and/or melanogenesis and/or at least one bluebuck variant associated with altered facial pattern.
Embodiment 2 is the transgenic animal of embodiment 1, wherein the at least one bluebuck gene variant associated with gray/blue coat color is selected from the group consisting of an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant.
Embodiment 2a is the transgenic animal of embodiment 1 or 2, wherein the at least one bluebuck gene variant associated with melanogenesis is selected from the group consisting of an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant.
Embodiment 3 is the transgenic animal of any one of embodiments 1-2a, wherein the at least one bluebuck gene variant associated with altered facial pattern is selected from the group consisting of an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.
Embodiment 4 is the transgenic animal of embodiment 1, wherein the bluebuck gene variant comprises at least one change in the nucleotide sequence of the gene.
Embodiment 5 is the transgenic animal of embodiment 4, wherein the change in the nucleotide sequence is a substitution, an insertion, a deletion, or a combination thereof.
Embodiment 6 is the transgenic animal of embodiment 5, wherein the substitution, the insertion, the deletion, or a combination thereof is in a 5′ untranslated region of the gene, an intron of the gene, an exon of the gene, a 3′ untranslated region of the gene, or a combination thereof.
Embodiment 7 is the transgenic animal of embodiment 5, wherein the substitution, the insertion, the deletion, or a combination thereof is in a regulatory region of the gene.
Embodiment 8 is the transgenic animal of embodiment 2, wherein the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof.
Embodiment 9 is the transgenic animal of embodiment 2, wherein the bluebuck gene variant associated with gray/blue coat color comprises a nucleotide sequence selected from the group consisting of SEQ ID NOs: 147-204 and combinations thereof.
Embodiment 9a is the transgenic animal of embodiment 2a, wherein the bluebuck gene variant associated with melanogenesis comprises a nucleotide sequence having at least 80%, at least 85%, at least 90%, or at least 95% identity to a nucleotide sequence selected from the group consisting of SEQ ID NOs: 48, 64, 68-72, 120-146, and combinations thereof.
Embodiment 9b is the transgenic animal of embodiment 2a, wherein the bluebuck gene variant associated with melanogenesis comprises a nucleotide sequence selected from the group consisting of SEQ ID NOs: 150, 166, 170-174, 222-248, and combinations thereof.
Embodiment 10 is the transgenic animal of embodiment 3, wherein the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof.
Embodiment 11 is the transgenic animal of embodiment 3, wherein the bluebuck gene variant associated with altered facial pattern comprises a nucleotide sequence selected from the group consisting of SEQ ID NOs: 162, 176, 178, 197, 205-221, and combinations thereof.
Embodiment 12 is the transgenic animal of embodiment 2, wherein the ASIP variant, the LYST variant, the SLC45A2 variant, the BCL2 variant, the DCT variant, and/or the MYO5A variant is in a coding region.
Embodiment 13 is the transgenic animal of embodiment 12, wherein:
(a) the ASIP variant in the coding region is selected from an isoleucine substitution for valine at amino acid position 39; and/or a tryptophan substitution for arginine at amino acid position 10, wherein the amino acid position corresponds to SEQ ID NO:2;
(b) the LYST variant in the coding region is selected from a tyrosine substitution for a cysteine at amino acid position 1352, and/or an arginine substitution for a valine at amino acid position 1079, wherein the amino acid position corresponds to SEQ ID NO:10;
(c) the SLC45A2 variant in the coding region is selected from a valine substitution for a methionine at amino acid position 475, and/or a cysteine substitution for an arginine at amino acid position 59, wherein the amino acid position corresponds to SEQ ID NO: 12;
(d) the BCL2 variant in the coding region is selected from a threonine substitution for an asparagine at amino acid position 139, and/or an aspartic acid substitution for a glycine at amino acid position 127, wherein the amino acid position corresponds to SEQ ID NO:14;
(e) the DCT variant in the coding region is selected from a phenylalanine substitution for a leucine at amino acid position 240, and/or an arginine substitution for a tryptophan at amino acid position 22, wherein the amino acid position corresponds to SEQ ID NO:16; and/or
(f) the MYO5A variant in the coding region is selected from a serine substitution for a proline at amino acid position 1321, and/or a serine substitution for an asparagine at amino acid position 372, wherein the amino acid position corresponds to SEQ ID NO:20.
Embodiment 14 is the transgenic animal of embodiment 2, wherein the ASIP variant, the MLPH variant, and/or the LYST variant is in a regulatory region.
Embodiment 15 is the transgenic animal of embodiment 14, wherein:
(a) the ASIP variant in the regulatory region is selected from an adenine substitution for a guanine at nucleotide position 919, a thymine substitution for a guanine at nucleotide position 1530, a thymine substitution for an adenine at nucleotide position 1578, a thymine substitution for a cytosine at nucleotide position 3703, an adenine substitution for a guanine at nucleotide position 3892, a cytosine substitution for a thymine at nucleotide position 4274, a thymine substitution for an adenine at nucleotide position 4370, a thymine substitution for a cytosine at nucleotide position 4807, a guanine substitution for an adenine at nucleotide position 4821, and/or an adenine substitution for a guanine at nucleotide position 4905, wherein the nucleotide position corresponds to SEQ ID NO:25;
(b) the MLPH variant in the regulatory region is selected from a cytosine substitution for a guanine at nucleotide position 1238, an adenine substitution for a thymine at nucleotide position 1299; an adenine substitution for a cytosine at nucleotide position 1488, an adenine substitution for a guanine at nucleotide position 1526, a guanine substitution for a thymine at nucleotide position 1618, an adenine substitution for a guanine at nucleotide position 1653, an adenine substitution for a guanine at nucleotide position 1672; a thymine substitution for a cytosine at nucleotide position 1718, an adenine substitution for a guanine at nucleotide position 1837, a guanine substitution for an adenine at nucleotide position 1949, an adenine substitution for a cytosine at nucleotide position 2472, a guanine substitution for an adenine at nucleotide position 2675, a thymine substitution for a cytosine at nucleotide position 2691, a guanine substitution for a thymine at nucleotide position 2900, a guanine substitution for an adenine at nucleotide position 3029, a guanine substitution for an adenine at nucleotide position 3090, a guanine substitution for a thymine at nucleotide position 3272, a cytosine substitution for a thymine at nucleotide position 3380, a cytosine substitution for a thymine at nucleotide position 3449, a cytosine substitution for a thymine at nucleotide position 3463, an adenine substitution for a guanine at nucleotide position 3513, a cytosine substitution for a thymine at nucleotide position 3559, a cytosine substitution for a thymine at nucleotide position 3581, a guanine substitution for a cytosine at nucleotide position 4352, a guanine substitution for a cytosine at nucleotide position 4356, a guanine substitution for an adenine at nucleotide position 4552, a guanine substitution for an adenine at nucleotide position 4690, a guanine substitution for an adenine at nucleotide position 4856, a cytosine substitution for a thymine at nucleotide position 4888, and/or a thymine substitution for a cytosine at nucleotide position 4990, wherein the nucleotide position corresponds to SEQ ID NO:27; and/or
(c) the LYST variant in the regulatory region is selected from a cytosine substitution for a guanine at nucleotide position 48, a cytosine substitution for an adenine at nucleotide position 67, a guanine substitution for a cytosine at nucleotide position 71, a thymine substitution for a cytosine at nucleotide position 247, a cytosine substitution for a thymine at nucleotide position 254, a guanine substitution for an adenine at nucleotide position 486, a thymine substitution for a cytosine at nucleotide position 883, a cytosine substitution for an adenine at nucleotide position 1002, an adenine substitution for a guanine at nucleotide position 1105, a thymine substitution for a cytosine at nucleotide position 1505, an adenine substitution for a guanine at nucleotide position 2350, a thymine substitution for a cytosine at nucleotide position 2612, a thymine substitution for a cytosine at nucleotide position 2661, a cytosine substitution for a thymine at nucleotide position 2668, a cytosine substitution for a thymine at nucleotide position 2818, a cytosine substitution for a guanine at nucleotide position 2873, a cytosine substitution for a guanine at nucleotide position 3223, an adenine substitution for a guanine at nucleotide position 3257, a thymine substitution for a cytosine at nucleotide position 4347, a cytosine substitution for a thymine at nucleotide position 4420, a thymine substitution for a cytosine at nucleotide position 4586, a cytosine substitution for a thymine at nucleotide position 4598, and/or a thymine substitution for a cytosine at nucleotide position 4619, wherein the nucleotide position corresponds to SEQ ID NO:29.
Embodiment 16 is the transgenic animal of embodiment 3, wherein the MITF variant and the KIT variant is in a regulatory region.
Embodiment 17 is the transgenic animal of any one of embodiments 16, wherein
(a) the MITF variant in the regulatory region is selected from a thymine substitution for a cytosine at nucleotide position 514, an adenine substitution for a guanine at nucleotide position 6733, a thymine substitution for an adenine at nucleotide position 21322, an adenine substitution for a thymine at nucleotide position 29009, a thymine substitution for a cytosine at nucleotide position 37136, an adenine substitution for a thymine adenine at nucleotide position 48433, a thymine substitution for a guanine at nucleotide position 50738, a thymine substitution for a guanine at nucleotide position 52771, a thymine substitution for an adenine at nucleotide position 53986, a guanine substitution for an adenine at nucleotide position 85470, an adenine substitution for a guanine at nucleotide position 85945, a guanine substitution for a thymine at nucleotide position 89390, an adenine substitution for a guanine at nucleotide position 91717, a cytosine substitution for an adenine at nucleotide position 110490, an adenine substitution for a guanine at nucleotide position 114903, an adenine substitution for a guanine at nucleotide position 116734, an adenine substitution for a guanine at nucleotide position 119238, a cytosine substitution for an adenine at nucleotide position 122369, an adenine substitution for a cytosine at nucleotide position 126635, an adenine substitution for a guanine at nucleotide position 129446, a thymine substitution for a guanine at nucleotide position 133318, an adenine substitution for a guanine at nucleotide position 138944, a cytosine substitution for a thymine at nucleotide position 150167, an adenine substitution for a cytosine at nucleotide position 187625, a guanine substitution for an adenine at nucleotide position 208940, an adenine substitution for a guanine at nucleotide position 211614, a guanine substitution for an adenine at nucleotide position 216326, and/or a cytosine substitution for a thymine at nucleotide position 217379, wherein the nucleotide position corresponds to SEQ ID NO:33; and/or
(b) the KIT variant in the regulatory region is selected from a thymine substitution for a cytosine at nucleotide position 268, a cytosine substitution for a guanine at nucleotide position 510, a guanine substitution for an adenine at nucleotide position 517, an adenine substitution for a guanine at nucleotide position 2108, a thymine substitution for a cytosine at nucleotide position 2401, an adenine substitution for a thymine at nucleotide position 2504, a cytosine substitution for a thymine at nucleotide position 2835, a guanine substitution for a adenine at nucleotide position 3365, a cytosine substitution for a thymine at nucleotide position 4044, an adenine substitution for a guanine at nucleotide position 4166, an adenine substitution for a cytosine at nucleotide position 4531, an adenine substitution for a guanine at nucleotide position 4569, a thymine substitution for a guanine at nucleotide position 4714, an adenine substitution for a cytosine at nucleotide position 4859, an adenine substitution for a guanine at nucleotide position 4892, and/or a guanine substitution for an adenine at nucleotide position 4907, wherein the nucleotide position corresponds to SEQ ID NO:31.
Embodiment 18 is the transgenic animal of any one of embodiments 1-17, wherein the transgenic animal further comprises at least one bluebuck gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.
Embodiment 19 is a transgenic animal comprising at least one bluebuck (Hippotragus leucophaeus) gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.
Embodiment 20 is the transgenic animal of embodiment 18 or 19, wherein:
(a) the loss-of-function ASIP variant comprises an insertion and deletion (indel);
(b) the DEFB103 missense variant comprises a glycine deletion at an amino acid position corresponding to position 23 of SEQ ID NO:4;
(c) the loss-of-function MLPH variant comprises an indel;
(d) the PMEL missense variant comprises a cysteine substitution for an arginine at an amino acid position corresponding to position 603 of SEQ ID NO:8;
(e) the loss-of-function LYST variant comprises an indel; and
(f) the loss-of-function SLC45A2 variant comprises an indel.
Embodiment 21 is the transgenic animal of any one of embodiments 1-19, wherein the animal comprises 2, 3, 4, 5, 6, 7, 8, 9, 10, 15, 20, 25, 30, 40, 50, 60, 70, 80, 90, 95, 100, 101, 102 (or any number in between) bluebuck gene variants.
Embodiment 22 is the transgenic animal of embodiment 21, wherein the bluebuck gene variant comprises one or more substitutions within the regulatory region and/or within the coding region of the gene as compared to a corresponding reference genome, wherein the corresponding reference genome is the genome of the animal prior to the one or more substitutions.
Embodiment 23 is the transgenic animal of embodiment 21, wherein the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RBI, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 24 is the transgenic animal of embodiment 23 wherein the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 25 is the transgenic animal of any one of embodiments 1-24, wherein the transgenic animal is an antelope.
Embodiment 26 is the transgenic animal of embodiment 25, wherein the transgenic animal is a roan antelope (Hippotragus equinus) cell or a sable antelope (Hippotragus niger).
Embodiment 27 is a recombinant host cell comprising at least one bluebuck (Hippotragus leucophaeus) gene variant associated with gray/blue coat color and/or melanogenesis and/or at least one bluebuck variant associated with altered facial pattern.
Embodiment 28 is the recombinant host cell of embodiment 27, wherein the at least one bluebuck gene variant associated with gray/blue coat color and/or melanogenesis is selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, and a WDFY1 variant.
Embodiment 29 is the recombinant host cell of embodiment 27, wherein the at least one bluebuck gene variant associated with altered facial pattern is selected from the group consisting of an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.
Embodiment 30 is the recombinant host cell of embodiment 27, wherein the bluebuck gene variant comprises at least one change in the nucleotide sequence of the gene.
Embodiment 31 is the recombinant host cell of embodiment 27, wherein the change in the nucleotide sequence is a substitution, an insertion, a deletion, or a combination thereof.
Embodiment 32 is the recombinant host cell of embodiment 31, wherein the substitution, the insertion, the deletion, or a combination thereof is in a 5′ untranslated region of the gene, an intron of the gene, an exon of the gene, a 3′ untranslated region of the gene, or a combination thereof.
Embodiment 33 is the recombinant host cell of embodiment 31, wherein the substitution, the insertion, the deletion, or a combination thereof is in a regulatory region of the gene.
Embodiment 34 is the recombinant host cell of embodiment 27, wherein the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof.
Embodiment 35 is the recombinant host cell of embodiment 27, wherein the bluebuck gene variant associated with gray/blue coat color comprises a nucleotide sequence selected from the group consisting of SEQ ID NOs: 147-204, and combinations thereof.
Embodiment 35a is the recombinant host cell of embodiment 27, wherein the bluebuck gene variant associated with melanogenesis comprises a nucleotide sequence having at least 80%, at least 85%, at least 90%, or at least 95% identity to a nucleotide sequence selected from the group consisting of SEQ ID NOs: 48, 64, 68-72, 120-146, and combinations thereof.
Embodiment 35b is the recombinant host cell of embodiment 27, wherein the bluebuck gene variant associated with melanogenesis comprises a nucleotide sequence selected from the group consisting of SEQ ID NOs: 150, 166, 170-174, 222-248, and combinations thereof.
Embodiment 36 is the recombinant host cell of embodiment 27, wherein the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof.
Embodiment 37 is the recombinant host cell of embodiment 27, wherein the bluebuck gene variant associated with altered facial pattern comprises a nucleotide sequence selected from the group consisting of SEQ ID NOs: 162, 176, 178, 197, 205-221, and combinations thereof.
Embodiment 38 is the recombinant host cell of embodiment 28, wherein the ASIP variant, the LYST variant, the SLC45A2 variant, the BCL2 variant, the DCT variant, and/or the MYO5A variant is in a coding region.
Embodiment 39 is the recombinant host cell of embodiment 38, wherein:
(a) the ASIP variant in the coding region is selected from an isoleucine substitution for valine at amino acid position 39; and/or a tryptophan substitution for arginine at amino acid position 10, wherein the amino acid position corresponds to SEQ ID NO:2;
(b) the LYST variant in the coding region is selected from a tyrosine substitution for a cysteine at amino acid position 1352, and/or an arginine substitution for a valine at amino acid position 1079, wherein the amino acid position corresponds to SEQ ID NO:10;
(c) the SLC45A2 variant in the coding region is selected from a valine substitution for a methionine at amino acid position 475, and/or a cysteine substitution for an arginine at amino acid position 59, wherein the amino acid position corresponds to SEQ ID NO: 12;
(d) the BCL2 variant in the coding region is selected from a threonine substitution for an asparagine at amino acid position 139, and/or an aspartic acid substitution for a glycine at amino acid position 127, wherein the amino acid position corresponds to SEQ ID NO: 14;
(e) the DCT variant in the coding region is selected from a phenylalanine substitution for a leucine at amino acid position 240, and/or an arginine substitution for a tryptophan at amino acid position 22, wherein the amino acid position corresponds to SEQ ID NO:16; and/or
(f) the MYO5A variant in the coding region is selected from a serine substitution for a proline at amino acid position 1321, and/or a serine substitution for an asparagine at amino acid position 372, wherein the amino acid position corresponds to SEQ ID NO:20.
Embodiment 40 is the recombinant host cell of embodiment 28, wherein the ASIP variant, the MLPH variant, and/or the LYST variant is in a regulatory region.
Embodiment 41 is the recombinant host cell of embodiment 40, wherein:
(a) the ASIP variant in the regulatory region is selected from an adenine substitution for a guanine at nucleotide position 919, a thymine substitution for a guanine at nucleotide position 1530, a thymine substitution for an adenine at nucleotide position 1578, a thymine substitution for a cytosine at nucleotide position 3703, an adenine substitution for a guanine at nucleotide position 3892, a cytosine substitution for a thymine at nucleotide position 4274, a thymine substitution for an adenine at nucleotide position 4370, a thymine substitution for a cytosine at nucleotide position 4807, a guanine substitution for an adenine at nucleotide position 4821, and/or an adenine substitution for a guanine at nucleotide position 4905, wherein the nucleotide position corresponds to SEQ ID NO:25;
(b) the MLPH variant in the regulatory region is selected from a cytosine substitution for a guanine at nucleotide position 1238, an adenine substitution for a thymine at nucleotide position 1299; an adenine substitution for a cytosine at nucleotide position 1488, an adenine substitution for a guanine at nucleotide position 1526, a guanine substitution for a thymine at nucleotide position 1618, an adenine substitution for a guanine at nucleotide position 1653, an adenine substitution for a guanine at nucleotide position 1672; a thymine substitution for a cytosine at nucleotide position 1718, an adenine substitution for a guanine at nucleotide position 1837, a guanine substitution for an adenine at nucleotide position 1949, an adenine substitution for a cytosine at nucleotide position 2472, a guanine substitution for an adenine at nucleotide position 2675, a thymine substitution for a cytosine at nucleotide position 2691, a guanine substitution for a thymine at nucleotide position 2900, a guanine substitution for an adenine at nucleotide position 3029, a guanine substitution for an adenine at nucleotide position 3090, a guanine substitution for a thymine at nucleotide position 3272, a cytosine substitution for a thymine at nucleotide position 3380, a cytosine substitution for a thymine at nucleotide position 3449, a cytosine substitution for a thymine at nucleotide position 3463, an adenine substitution for a guanine at nucleotide position 3513, a cytosine substitution for a thymine at nucleotide position 3559, a cytosine substitution for a thymine at nucleotide position 3581, a guanine substitution for a cytosine at nucleotide position 4352, a guanine substitution for a cytosine at nucleotide position 4356, a guanine substitution for an adenine at nucleotide position 4552, a guanine substitution for an adenine at nucleotide position 4690, a guanine substitution for an adenine at nucleotide position 4856, a cytosine substitution for a thymine at nucleotide position 4888, and/or a thymine substitution for a cytosine at nucleotide position 4990, wherein the nucleotide position corresponds to SEQ ID NO:27; and/or
(c) the LYST variant in the regulatory region is selected from a cytosine substitution for a guanine at nucleotide position 48, a cytosine substitution for an adenine at nucleotide position 67, a guanine substitution for a cytosine at nucleotide position 71, a thymine substitution for a cytosine at nucleotide position 247, a cytosine substitution for a thymine at nucleotide position 254, a guanine substitution for an adenine at nucleotide position 486, a thymine substitution for a cytosine at nucleotide position 883, a cytosine substitution for an adenine at nucleotide position 1002, an adenine substitution for a guanine at nucleotide position 1105, a thymine substitution for a cytosine at nucleotide position 1505, an adenine substitution for a guanine at nucleotide position 2350, a thymine substitution for a cytosine at nucleotide position 2612, a thymine substitution for a cytosine at nucleotide position 2661, a cytosine substitution for a thymine at nucleotide position 2668, a cytosine substitution for a thymine at nucleotide position 2818, a cytosine substitution for a guanine at nucleotide position 2873, a cytosine substitution for a guanine at nucleotide position 3223, an adenine substitution for a guanine at nucleotide position 3257, a thymine substitution for a cytosine at nucleotide position 4347, a cytosine substitution for a thymine at nucleotide position 4420, a thymine substitution for a cytosine at nucleotide position 4586, a cytosine substitution for a thymine at nucleotide position 4598, and/or a thymine substitution for a cytosine at nucleotide position 4619, wherein the nucleotide position corresponds to SEQ ID NO:29.
Embodiment 42 is the recombinant host cell of embodiment 29, wherein the MITF variant and the KIT variant is in a regulatory region.
Embodiment 43 is the recombinant host cell of embodiment 42, wherein
(a) the MITF variant in the regulatory region is selected from a thymine substitution for a cytosine at nucleotide position 514, an adenine substitution for a guanine at nucleotide position 6733, a thymine substitution for an adenine at nucleotide position 21322, an adenine substitution for a thymine at nucleotide position 29009, a thymine substitution for a cytosine at nucleotide position 37136, an adenine substitution for a thymine adenine at nucleotide position 48433, a thymine substitution for a guanine at nucleotide position 50738, a thymine substitution for a guanine at nucleotide position 52771, a thymine substitution for an adenine at nucleotide position 53986, a guanine substitution for an adenine at nucleotide position 85470, an adenine substitution for a guanine at nucleotide position 85945, a guanine substitution for a thymine at nucleotide position 89390, an adenine substitution for a guanine at nucleotide position 91717, a cytosine substitution for an adenine at nucleotide position 110490, an adenine substitution for a guanine at nucleotide position 114903, an adenine substitution for a guanine at nucleotide position 116734, an adenine substitution for a guanine at nucleotide position 119238, a cytosine substitution for an adenine at nucleotide position 122369, an adenine substitution for a cytosine at nucleotide position 126635, an adenine substitution for a guanine at nucleotide position 129446, a thymine substitution for a guanine at nucleotide position 133318, an adenine substitution for a guanine at nucleotide position 138944, a cytosine substitution for a thymine at nucleotide position 150167, an adenine substitution for a cytosine at nucleotide position 187625, a guanine substitution for an adenine at nucleotide position 208940, an adenine substitution for a guanine at nucleotide position 211614, a guanine substitution for an adenine at nucleotide position 216326, and/or a cytosine substitution for a thymine at nucleotide position 217379, wherein the nucleotide position corresponds to SEQ ID NO:33; and/or
(b) the KIT variant in the regulatory region is selected from a thymine substitution for a cytosine at nucleotide position 268, a cytosine substitution for a guanine at nucleotide position 510, a guanine substitution for an adenine at nucleotide position 517, an adenine substitution for a guanine at nucleotide position 2108, a thymine substitution for a cytosine at nucleotide position 2401, an adenine substitution for a thymine at nucleotide position 2504, a cytosine substitution for a thymine at nucleotide position 2835, a guanine substitution for a adenine at nucleotide position 3365, a cytosine substitution for a thymine at nucleotide position 4044, an adenine substitution for a guanine at nucleotide position 4166, an adenine substitution for a cytosine at nucleotide position 4531, an adenine substitution for a guanine at nucleotide position 4569, a thymine substitution for a guanine at nucleotide position 4714, an adenine substitution for a cytosine at nucleotide position 4859, an adenine substitution for a guanine at nucleotide position 4892, and/or a guanine substitution for an adenine at nucleotide position 4907, wherein the nucleotide position corresponds to SEQ ID NO:31.
Embodiment 44 is the recombinant host cell of embodiment 27, wherein the recombinant host cell further comprises at least one bluebuck gene variant with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.
Embodiment 45 is a recombinant host cell comprising at least one bluebuck (Hippotragus leucophaeus) gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.
Embodiment 46 is the recombinant host cell of embodiment 44 or 45, wherein:
(a) the loss-of-function ASIP variant comprises an insertion and deletion (indel);
(b) the DEFB103 missense variant comprises a glycine deletion at an amino acid position corresponding to position 23 of SEQ ID NO:4;
(c) the loss-of-function MLPH variant comprises an indel;
(d) the PMEL missense variant comprises a cysteine substitution for an arginine at an amino acid position corresponding to position 603 of SEQ ID NO:8;
(e) the loss-of-function LYST variant comprises an indel; and
(f) the loss-of-function SLC45A2 variant comprises an indel.
Embodiment 47 is the recombinant host cell of claim 45, wherein the recombinant host cell comprises 2, 3, 4, 5, 6, 7, 8, 9, 10, 15, 20, 25, 30, 40, 50, 60, 70, 80, 90, 95, 100, 101, 102 (or any number in between) bluebuck gene variants.
Embodiment 48 is the recombinant host cell of embodiment 45, wherein the recombinant host cell fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 49 is the recombinant host cell of embodiment 48, wherein the recombinant host cell fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 50 is the recombinant host cell of any one of embodiments 27-49, wherein the recombinant host cell is an antelope cell.
Embodiment 51 is the recombinant host cell of embodiment 50, wherein the recombinant host cell is a roan antelope (Hippotragus equinus) cell or a sable antelope (Hippotragus niger) cell.
Embodiment 52 is the recombinant host cell of any one of embodiments 27-49, wherein the recombinant host cell is a stem cell.
Embodiment 53 is the recombinant host cell of embodiment 52, wherein the stem cell is selected from an induced stem cell, an embryonic stem (ES) cell, or a mesenchymal stem cell (MSC).
Embodiment 54 is the recombinant host cell of any one of embodiments 27-49, wherein the recombinant host cell is a reprogrammed cell.
Embodiment 55 is the recombinant host cell of any one of embodiments 27-49, wherein the recombinant host cell is a fibroblast cell or a mesenchymal cell.
Embodiment 56 is the recombinant host cell of any one of embodiments 27-49, wherein the recombinant host cell is an endothelial progenitor cell (EPC) or a pericyte.
Embodiment 57 is the recombinant host cell of any one of embodiments 27-49, wherein the recombinant host cell is selected from the group consisting of a nerve cell, a cartilage cell, a bone cell, a muscle cell, a fat cell, and an epidermal cell.
Embodiment 58 is a method of creating a transgenic animal with gray/blue coat color and/or an altered melanin biosynthesis pathway of a bluebuck and/or an altered facial pattern of a bluebuck, the method comprising:
(a) obtaining a cell from the animal;
(b) introducing into the cell at least one bluebuck (Hippotragus leucophaeus) gene variant associated with gray/blue coat color and/or melanogenesis, wherein the one or more bluebuck gene variants associated with gray/blue coat color are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MPLH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant, and the one or more bluebuck gene variants associated with melanogenesis are selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant; and/or at least one bluebuck (Hippotragus leucophaeus) gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant; and
(c) utilizing the recombinant cell to produce a transgenic animal,
   wherein the transgenic animal has a gray/blue coat color and/or an altered melanin biosynthesis pathway of a bluebuck and/or an altered facial pattern of a bluebuck.
Embodiment 59 is the method of embodiment 58, wherein the bluebuck gene variant comprises at least one change in the nucleotide sequence of the gene.
Embodiment 60 is the method of embodiment 59, wherein the change in the nucleotide sequence is a substitution, an insertion, a deletion, or a combination thereof.
Embodiment 61 is the method of embodiment 60, wherein the substitution, the insertion, the deletion, or a combination thereof is in a 5′ untranslated region of the gene, an intron of the gene, an exon of the gene, a 3′ untranslated region of the gene, or a combination thereof.
Embodiment 62 is the method of embodiment 60, wherein the substitution, the insertion, the deletion, or a combination thereof is in a regulatory region of the gene.
Embodiment 63 is the method of embodiment 58, wherein the bluebuck gene variant associated with gray/blue coat color comprises a nucleotide sequence having at least 80%, at least 85%, at least 90%, or at least 95% identity to a nucleotide sequence selected from the group consisting of SEQ ID NOs: 45-102 and combinations thereof.
Embodiment 64 is the method of embodiment 58, wherein the bluebuck gene variant associated with gray/blue coat color comprises a nucleotide sequence selected from the group consisting of SEQ ID NOs: 147-204, and combinations thereof.
Embodiment 64a is the method of embodiment 58, wherein the bluebuck gene variant associated with melanogenesis comprises a nucleotide sequence having at least 80%, at least 85%, at least 90%, or at least 95% identity to a nucleotide sequence selected from the group consisting of SEQ ID NOs: 48, 64, 68-72, 120-146, and combinations thereof.
Embodiment 64 is the method of embodiment 58, wherein the bluebuck gene variant associated with melanogenesis comprises a nucleotide sequence selected from the group consisting of SEQ ID NOs: 150, 166, 170-174, 222-248, and combinations thereof.
Embodiment 65 is the method of embodiment 58, wherein the bluebuck gene variant associated with altered facial pattern comprises a nucleotide sequence having at least 80%, at least 85%, at least 90%, or at least 95% identity to a nucleotide sequence selected from the group consisting of SEQ ID NOs: 60, 74, 76, 95, 103-119, and combinations thereof.
Embodiment 66 is the method of embodiment 58, wherein the bluebuck gene variant associated with altered facial pattern comprises a nucleotide sequence selected from the group consisting of SEQ ID NOs: 162, 176, 178, 197, 205-221, and combinations thereof.
Embodiment 67 is the method of any one of embodiments 58, wherein the ASIP variant, the LYST variant, the SLC45A2 variant, the BCL2 variant, the DCT variant, and/or the MYO5A variant is in a coding region.
Embodiment 68 is the method of embodiment 67, wherein:
(a) the ASIP variant in the coding region is selected from an isoleucine substitution for valine at amino acid position 39; and/or a tryptophan substitution for arginine at amino acid position 10, wherein the amino acid position corresponds to SEQ ID NO:2;
(b) the LYST variant in the coding region is selected from a tyrosine substitution for a cysteine at amino acid position 1352, and/or an arginine substitution for a valine at amino acid position 1079, wherein the amino acid position corresponds to SEQ ID NO:10;
(c) the SLC45A2 variant in the coding region is selected from a valine substitution for a methionine at amino acid position 475, and/or a cysteine substitution for an arginine at amino acid position 59, wherein the amino acid position corresponds to SEQ ID NO:12;
(d) the BCL2 variant in the coding region is selected from a threonine substitution for an asparagine at amino acid position 139, and/or an aspartic acid substitution for a glycine at amino acid position 127, wherein the amino acid position corresponds to SEQ ID NO:14;
(e) the DCT variant in the coding region is selected from a phenylalanine substitution for a leucine at amino acid position 240, and/or an arginine substitution for a tryptophan at amino acid position 22, wherein the amino acid position corresponds to SEQ ID NO:16; and/or
(f) the MYO5A variant in the coding region is selected from a serine substitution for a proline at amino acid position 1321, and/or a serine substitution for an asparagine at amino acid position 372, wherein the amino acid position corresponds to SEQ ID NO:20.
Embodiment 69 is the method of embodiment 58, wherein the ASIP variant, the MLPH variant, and/or the LYST variant is in a regulatory region.
Embodiment 70 is the method of embodiment 69, wherein:
(a) the ASIP variant in the regulatory region is selected from an adenine substitution for a guanine at nucleotide position 919, a thymine substitution for a guanine at nucleotide position 1530, a thymine substitution for an adenine at nucleotide position 1578, a thymine substitution for a cytosine at nucleotide position 3703, an adenine substitution for a guanine at nucleotide position 3892, a cytosine substitution for a thymine at nucleotide position 4274, a thymine substitution for an adenine at nucleotide position 4370, a thymine substitution for a cytosine at nucleotide position 4807, a guanine substitution for an adenine at nucleotide position 4821, and/or an adenine substitution for a guanine at nucleotide position 4905, wherein the nucleotide position corresponds to SEQ ID NO:25;
(b) the MLPH variant in the regulatory region is selected from a cytosine substitution for a guanine at nucleotide position 1238, an adenine substitution for a thymine at nucleotide position 1299; an adenine substitution for a cytosine at nucleotide position 1488, an adenine substitution for a guanine at nucleotide position 1526, a guanine substitution for a thymine at nucleotide position 1618, an adenine substitution for a guanine at nucleotide position 1653, an adenine substitution for a guanine at nucleotide position 1672; a thymine substitution for a cytosine at nucleotide position 1718, an adenine substitution for a guanine at nucleotide position 1837, a guanine substitution for an adenine at nucleotide position 1949, an adenine substitution for a cytosine at nucleotide position 2472, a guanine substitution for an adenine at nucleotide position 2675, a thymine substitution for a cytosine at nucleotide position 2691, a guanine substitution for a thymine at nucleotide position 2900, a guanine substitution for an adenine at nucleotide position 3029, a guanine substitution for an adenine at nucleotide position 3090, a guanine substitution for a thymine at nucleotide position 3272, a cytosine substitution for a thymine at nucleotide position 3380, a cytosine substitution for a thymine at nucleotide position 3449, a cytosine substitution for a thymine at nucleotide position 3463, an adenine substitution for a guanine at nucleotide position 3513, a cytosine substitution for a thymine at nucleotide position 3559, a cytosine substitution for a thymine at nucleotide position 3581, a guanine substitution for a cytosine at nucleotide position 4352, a guanine substitution for a cytosine at nucleotide position 4356, a guanine substitution for an adenine at nucleotide position 4552, a guanine substitution for an adenine at nucleotide position 4690, a guanine substitution for an adenine at nucleotide position 4856, a cytosine substitution for a thymine at nucleotide position 4888, and/or a thymine substitution for a cytosine at nucleotide position 4990, wherein the nucleotide position corresponds to SEQ ID NO:27; and/or
(c) the LYST variant in the regulatory region is selected from a cytosine substitution for a guanine at nucleotide position 48, a cytosine substitution for an adenine at nucleotide position 67, a guanine substitution for a cytosine at nucleotide position 71, a thymine substitution for a cytosine at nucleotide position 247, a cytosine substitution for a thymine at nucleotide position 254, a guanine substitution for an adenine at nucleotide position 486, a thymine substitution for a cytosine at nucleotide position 883, a cytosine substitution for an adenine at nucleotide position 1002, an adenine substitution for a guanine at nucleotide position 1105, a thymine substitution for a cytosine at nucleotide position 1505, an adenine substitution for a guanine at nucleotide position 2350, a thymine substitution for a cytosine at nucleotide position 2612, a thymine substitution for a cytosine at nucleotide position 2661, a cytosine substitution for a thymine at nucleotide position 2668, a cytosine substitution for a thymine at nucleotide position 2818, a cytosine substitution for a guanine at nucleotide position 2873, a cytosine substitution for a guanine at nucleotide position 3223, an adenine substitution for a guanine at nucleotide position 3257, a thymine substitution for a cytosine at nucleotide position 4347, a cytosine substitution for a thymine at nucleotide position 4420, a thymine substitution for a cytosine at nucleotide position 4586, a cytosine substitution for a thymine at nucleotide position 4598, and/or a thymine substitution for a cytosine at nucleotide position 4619, wherein the nucleotide position corresponds to SEQ ID NO:29.
Embodiment 71 is the method of embodiment 58, wherein the transgenic animal further comprises at least one bluebuck gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.
Embodiment 72 is a method of creating a transgenic animal with gray/blue coat color, the method comprising:
(a) obtaining a cell from the animal;
(b) introducing into the cell at least bluebuck gene variant associated with gray/blue coat color selected from:
   (1) a loss-of-function agouti signaling protein (ASIP) variant;
   (2) a defensin beta 103 (DEFB103) missense variant;
   (3) a loss-of-function melanophilin (MLPH) variant;
   (4) a premelanosome protein (PMEL) missense variant;
   (5) a loss-of-function lysosomal trafficking regulator (LYST) variant; and/or
   (6) a loss-of-function solute carrier family 45 member 2 (SLC45A2) variant, whereby introducing the at least one bluebuck gene variant produces a recombinant cell; and
(c) utilizing the recombinant cell to produce a transgenic animal,
wherein the transgenic animal has a gray/blue coat color.
Embodiment 73 is the method of embodiments 71 or 72, wherein:
(a) the loss-of-function ASIP variant comprises an insertion and deletion (indel);
(b) the DEFB103 missense variant comprises a glycine deletion at an amino acid position corresponding to position 23 of SEQ ID NO:4;
(c) the loss-of-function MLPH variant comprises an indel;
(d) the PMEL missense variant comprises a cysteine substitution for an arginine at an amino acid position corresponding to position 603 of SEQ ID NO:8;
(e) the loss-of-function LYST variant comprises an indel; and
(f) the loss-of-function SLC45A2 variant comprises an indel.
Embodiment 74 is the method of embodiment 73, wherein the transgenic animal further comprises at least one bluebuck (Hippotragus leucophaeus) gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.
Embodiment 75 is the method of embodiment 58 or 74, wherein the MITF variant and the KIT variant is in a regulatory region.
Embodiment 76 is the method of embodiment 75, wherein
(a) the MITF variant in the regulatory region is selected from a thymine substitution for a cytosine at nucleotide position 514, an adenine substitution for a guanine at nucleotide position 6733, a thymine substitution for an adenine at nucleotide position 21322, an adenine substitution for a thymine at nucleotide position 29009, a thymine substitution for a cytosine at nucleotide position 37136, an adenine substitution for a thymine adenine at nucleotide position 48433, a thymine substitution for a guanine at nucleotide position 50738, a thymine substitution for a guanine at nucleotide position 52771, a thymine substitution for an adenine at nucleotide position 53986, a guanine substitution for an adenine at nucleotide position 85470, an adenine substitution for a guanine at nucleotide position 85945, a guanine substitution for a thymine at nucleotide position 89390, an adenine substitution for a guanine at nucleotide position 91717, a cytosine substitution for an adenine at nucleotide position 110490, an adenine substitution for a guanine at nucleotide position 114903, an adenine substitution for a guanine at nucleotide position 116734, an adenine substitution for a guanine at nucleotide position 119238, a cytosine substitution for an adenine at nucleotide position 122369, an adenine substitution for a cytosine at nucleotide position 126635, an adenine substitution for a guanine at nucleotide position 129446, a thymine substitution for a guanine at nucleotide position 133318, an adenine substitution for a guanine at nucleotide position 138944, a cytosine substitution for a thymine at nucleotide position 150167, an adenine substitution for a cytosine at nucleotide position 187625, a guanine substitution for an adenine at nucleotide position 208940, an adenine substitution for a guanine at nucleotide position 211614, a guanine substitution for an adenine at nucleotide position 216326, and/or a cytosine substitution for a thymine at nucleotide position 217379, wherein the nucleotide position corresponds to SEQ ID NO:33; and/or
(b) the KIT variant in the regulatory region is selected from a thymine substitution for a cytosine at nucleotide position 268, a cytosine substitution for a guanine at nucleotide position 510, a guanine substitution for an adenine at nucleotide position 517, an adenine substitution for a guanine at nucleotide position 2108, a thymine substitution for a cytosine at nucleotide position 2401, an adenine substitution for a thymine at nucleotide position 2504, a cytosine substitution for a thymine at nucleotide position 2835, a guanine substitution for a adenine at nucleotide position 3365, a cytosine substitution for a thymine at nucleotide position 4044, an adenine substitution for a guanine at nucleotide position 4166, an adenine substitution for a cytosine at nucleotide position 4531, an adenine substitution for a guanine at nucleotide position 4569, a thymine substitution for a guanine at nucleotide position 4714, an adenine substitution for a cytosine at nucleotide position 4859, an adenine substitution for a guanine at nucleotide position 4892, and/or a guanine substitution for an adenine at nucleotide position 4907, wherein the nucleotide position corresponds to SEQ ID NO:31.
Embodiment 77 is the method of embodiment 71, wherein the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 78 is the method of embodiment 77, wherein the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MPLH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.
Embodiment 79 is the method of any one of embodiments 58-78, wherein the cell is a stem cell.
Embodiment 80 is the method of embodiment 79, wherein the stem cell is selected from an induced stem cell, an embryonic stem (ES) cell, or a mesenchymal stem cell (MSC).
Embodiment 81 is the method of any one of embodiments 58-78, wherein the cell is a reprogrammed cell.
Embodiment 82 is the method of any one of embodiments 58-78, wherein the cell is a fibroblast cell or a mesenchymal cell.
Embodiment 83 is the method of any one of embodiments 58-78, wherein the cell is an endothelial cell.
Embodiment 84 is the method of any one of embodiments 58-78, wherein the cell is selected from the group consisting of a nerve cell, a cartilage cell, a bone cell, a muscle cell, a fat cell, and an epidermal cell.
Embodiment 85 is the method of any one of embodiments 58-84, wherein the animal is an antelope.
Embodiment 86 is the method of embodiment 85, wherein the antelope is selected from a roan antelope (Hippotragus equimuis) or a sable antelope (Hippotragus niger).
Embodiment 87 is a transgenic animal made by the method of any one of embodiments 58-86.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### EXAMPLES

### Example 1: Creation of Cells with Knock Outs in ASIP, MLPH, LYST, SLC45A2 and MITF Genes

### Reagents

IDT Alt-R S.p. Cas9 EGFP RNP (Catalog number: 10008161); A 1 μg/μl working stock was prepared according to standard protocols.
ASIP gRNA: sqRNA sequence: ACACCAGCAAGGUAGCCAGG (SEQ ID NO:37). A 15 μM working stock of the ASIP gRNA was prepared according to standard protocols.
MLPH gRNA: sqRNA sequence: AGAGGCCAAGCACGUCUGGG (SEQ ID NO:38). A 15 μM working stock of the MPLH gRNA was prepared according to standard protocols.
LYST gRNA: sqRNA sequence: CUGAAACAGAGCUUGCACAG (SEQ ID NO:39). A 15 μM working stock of the LYST gRNA was prepared according to standard protocols.
SLC45A2 gRNA: sqRNA sequence: GGAAGGCAGUCCAUCCAAUG (SEQ ID NO: 40). A 15 μM working stock of the SLC45A2 gRNA was prepared according to standard protocols.
MITF gRNAs: sqRNA sequences: MITF-L1: TCATAGCTACTCCCACAGGG (SEQ ID NO: 249); MITF-L2: TGGTGATTCCTTGAGGGATG (SEQ ID NO:250); MITF-L3: AACTAAGTAAGCTTGCCCTG (SEQ ID NO:251); MITF-R1: TTACCCACATCACTAAG ACC (SEQ ID NO:252); MITF-R2: TAAGGCGCTGGTGAAGTCAG (SEQ ID NO:253); MITF-R3: ACCAATCCTCACCTTTCACG (SEQ ID NO:254); MITF-R4: TCCTAAACGTACAATGA CTG (SEQ ID NO:255). 15 μM working stocks of the MITF gRNAs were prepared according to standard protocols.

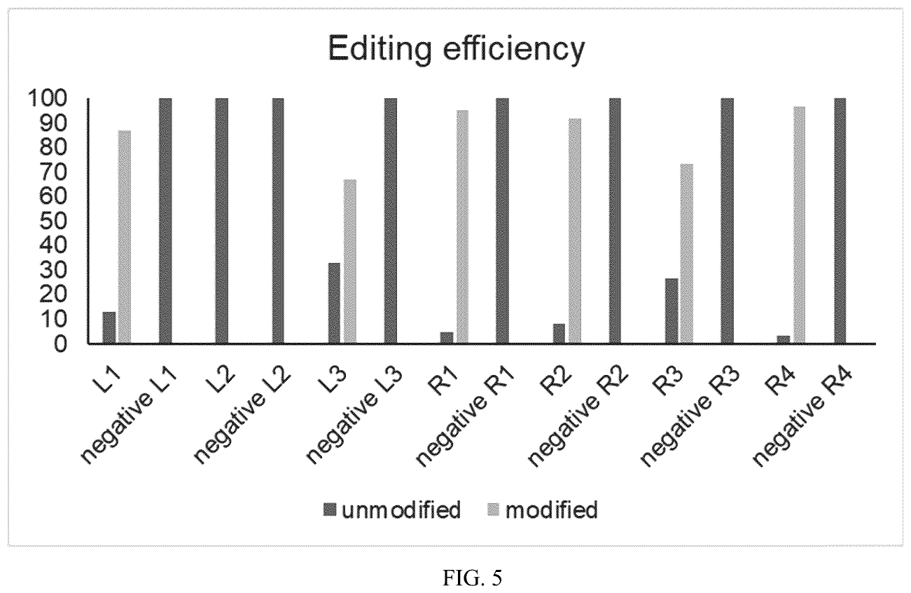

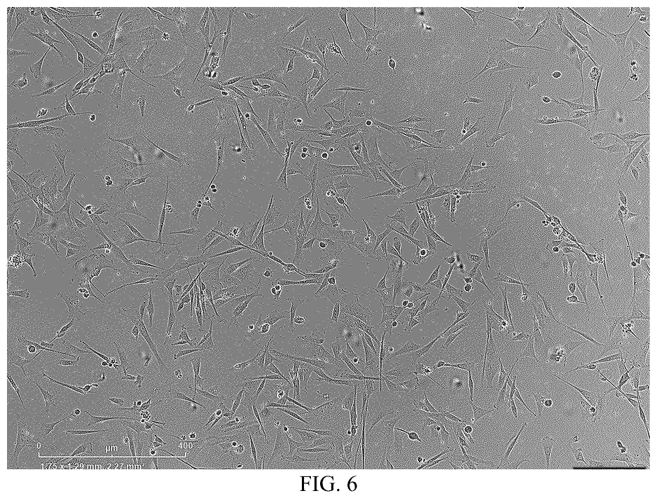

In order to generate regulatory edits in the MITF locus that were predicted to lead to changes in coat patterning, homology directed repair was used to incorporate these variants. The ability to cut at the MITF locus was tested by testing the editing efficiency of 7 different gRNAs (FIG. 5). Editing efficiency was seen to be consistently high in all cases except for one, suggesting further edits will be able to be generated at this locus. Furthermore, delivery of editing materials was observed in roan fibroblasts (FIG. 6) via GFP fluorescence imaging.

For RNP and mRNA, the following chemistries were used: Ribojuice, RNAiMAX, and CRISPRmax. These were run in parallel for both mRNA and RNP Cas9. Duplicates were run of the above conditions so that 2 biological replicates were created for each gene.

Additional reagents: T175 (or larger) flask; DMEM media (antibiotic free); Opti-MEM; 1× DPBS; 0.5% Trypsin-EDTA; pipettes and tips; cell counter; trypan blue; 15 mL conical tubes; 50 mL conical tubes; microcentrifuge tubes; CRISPRmax; IDT Alt-R spCas9 GFP RNP (1 mg/ml); and guide RNA stocks disclosed above.

Cell Culture preparation: Cells were grown in a T-175 to attain the minimum total cells required of 1.9×10⁶ cells. Once the cells reached 60+% confluence, the cells were harvested for transfection. To harvest the cells, (1) the media was aspirated off of the T-175 flask on the opposing side of the adherent cells; (2) any remaining media was washed out of the T-175 flask with 25 mL of 1× DPBS, and then the DPBS was aspirated out of the flask; (3) approximately 7 mL of 0.05% Trypsin-EDTA was added to the flask, and the flask was placed back in the incubator for 2-3 minutes; (4) after the time had elapsed, the flask was removed from the incubator and 21 mL of cDMEM was added to neutralize the Trypsin reaction; (5) all of the media and cells was pipetted out of the flask and placed in a 50 ml conical; (6) the cells were centrifuged at 300×g for 5 minutes; (7) after the cells were pelleted, the cells were resuspended in 2 mL cDMEM; 20 μL were removed into a micro centrifuge tube and 20 μL of Trypan Blue was added to the micro centrifuge tube; 10 μL was added to each chamber of the cell counter and the cell density total was collected; (8) from this amount of cells to remove to achieve the needed cell total was calculated and these cells were set aside into a 50 ml conical for transfection, and any remaining cells were used for expansion or freezing. Cells used for this experiment were 23HE004 FB.

CRISPRmax: For 2 reactions each: 6 pmol RNAi duplex were diluted in 50 μl Opti-MEM I Medium without serum in the well of the tissue culture plate or microcentrifuge tube. The solution was mixed gently. For GFP RNP, 1.5 μL (6 pmol) was added; For ASIP RNP: 1250 ng: 240 ng: 2.5 μL Cas9 RNP and 6.66 μL ASIP guide was added. There was 1 reaction only of ASIP GFP mRNA: 1.25 μL Cas9 mRNA and 3.33 μL ASIP guide. Cas9 Plus reagent was mixed gently, then 2.5 μL of Cas9 Plus Reagent was added to 2× GFP conditions and 5.0 μL of Cas9 Plus Reagent was added to each of the mRNA and RNP reactions. CrisprMax reagent was mixed gently, then 1.5 μL of CrisprMax Reagent was added to 2× GFP conditions and 3.0 μL of CrisprMax Reagent was added to each of the mRNA and RNP reactions. All solutions were titurated/mixed well and were allowed to stand for 10 minutes to let the complexes form. 1.0 mL of 100 k cells was added and plated into 2 replicate wells. The cells were incubated for 24 hours for GFP then run through Flow. The ASIP cells were incubated for 48 hours expanding as necessary and then counted and pelleted to determine doubling rate and toxicity.

Negative control: For the negative control, there were 50K cells per well. There were 2 wells of negative controls. 0.5 mL of cells were added to the plate and no additional gRNA was added to the cells.

### Example 2: Creation of Roan Fibroblast Monoclonal Cells Lines Containing Knock Outs for ASIP, MLPH, LYST, and SLC45A2 Genes

Reagents: Seeded, proliferating fibroblasts in regular growth media; FGF2 (recombinant human FGF); 96 well plates; 100 ml multiwell channel pipettor; 25 ml well boats; 200 ml filter pipettes; 5 ml FACS tubes; FACS buffer (PBS, —Mg, —Ca; 2% FBS; 2.5 mM EDTA, pH 8.0; 25 mM HEPES; 1% Penn/Strep).
Cell information: Roan antelope cells (23HE_FB_004_Passage 5).
Preparation for Cell Sorting: Two days before the sort, the flask was seeded with appropriate number of cells to reach 80% confluency by sort day. The maximum amount of media (with a buffer to avoid spillage) was plated in the flask. The day before the sort, the media was collected from the cells to be sorted. The media was stored for filtration the day of sort. The cells were fed with fresh media to ensure that the cells were growing appropriately.

The day of the sort, the media was prepared as follows: The previously collected media was obtained and filtered 1:1 with fresh media in a 0.22 μM filter flask. Additional FBS was added to the media to bring to at least 20%. If using fibroblasts, FGF2 was added to the media for an effective concentration of 10 ng/ml (4.08 μL). Approximately 10.5 mL/96 well plate. The FACS buffer was cold and remained cold throughout the experiment.

The date of the sort, the plates were prepared as follows: Before the sort begins, 100 μL of conditioned media made above was pipetted and stored at 37° C. until the experiment was ready.

To sort the cells, the media was warmed to start the process. The start-up process for the Sony machine was begun. Once the machine had gone through the start-up process or if the machine had been cleaned, the cells were harvested for sort. The appropriate cell population was trypsinized/accutased with 0.05% trypsin until the cells came off the flask/plate at 37° C. While cells were in trypsin, an ice bucket was obtained, and the FACS tubes were numbered for the sort and tubes were obtained to spin down the cells. Once the cells detached, the trypsin was neutralized with regular media, and spun down at 500g×10 min. The media was removed from the cell pellet or as close as possible without disturbing the cell pellet. The pellet was resuspended in FACS buffer and pipetted through the top of the FACS tubes. The cells were pipetted to remove clumps of cells. The cells were placed back on ice. Once the cells were obtained, the cells were sorted. The gates for FACS were set, and the cells were gathered. Once the cells have been sorted, the cells were collected and placed at 37° C. The cells were stored at 37° C. as soon as the sort was finished. The cells were observed 24 hours later to confirm no contamination had occurred.

### Results:

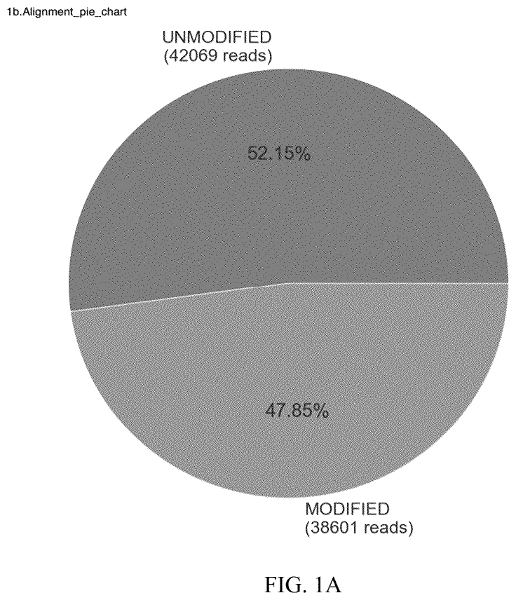

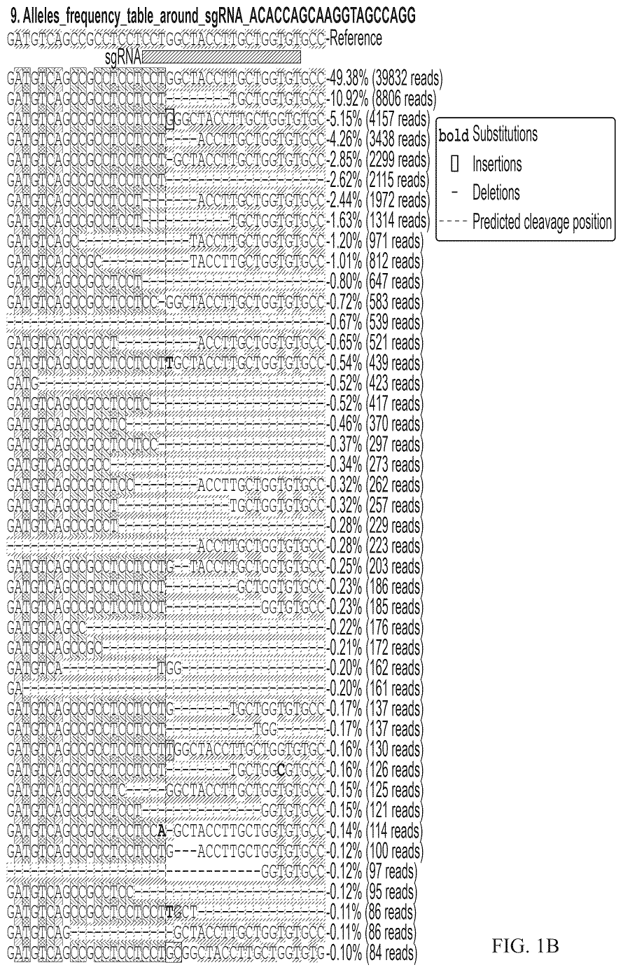

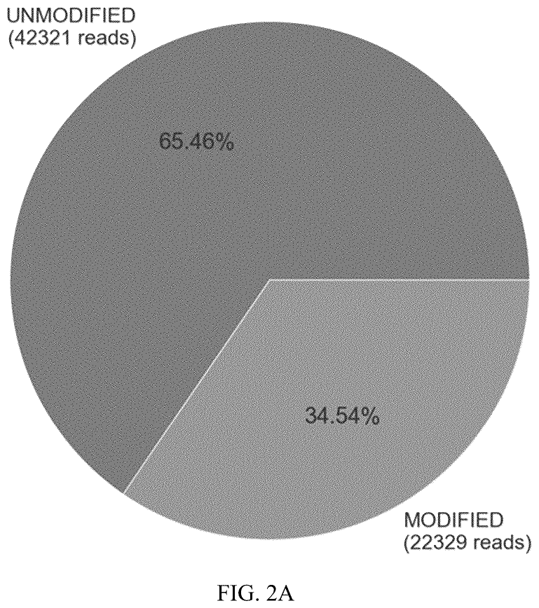

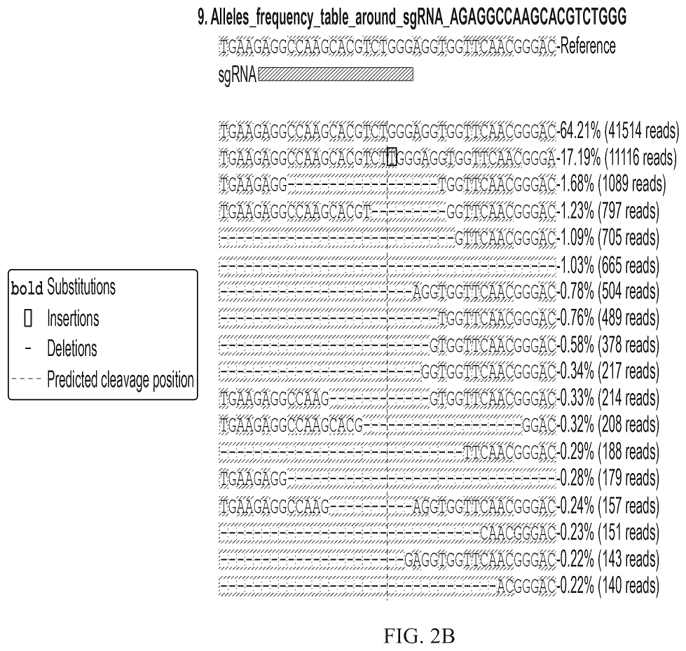

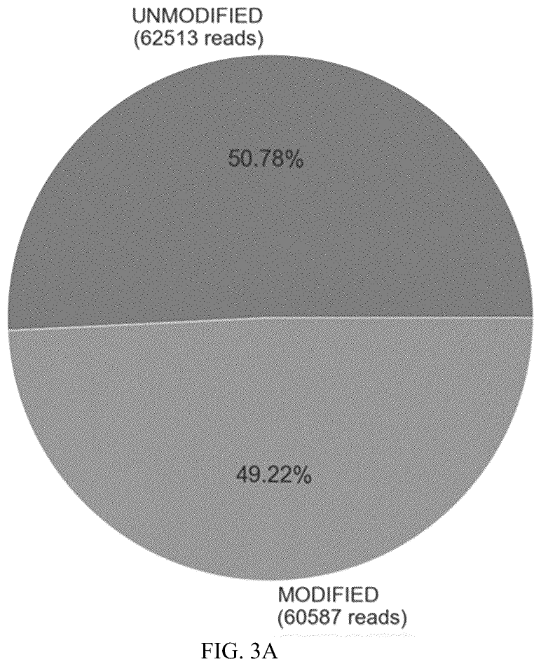

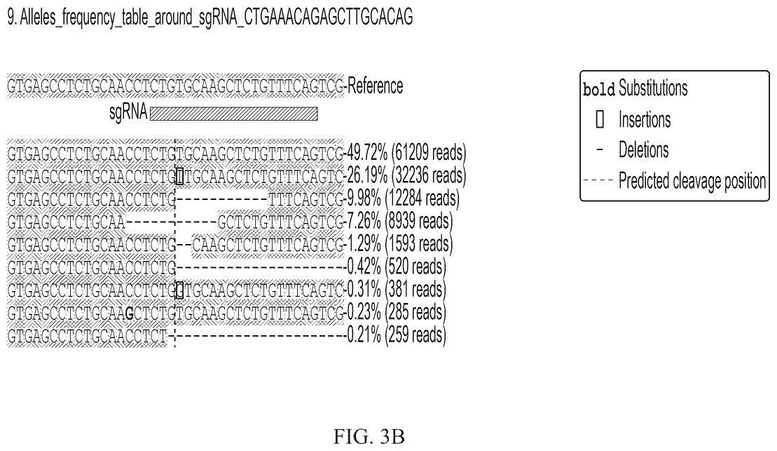

Genomic Analysis: In order to determine how to revive the blue antelope (Hippotragus leucophaeus), the genome of the blue antelope was analyzed for variants. Variants found in the blue antelope genome in genes known to be associated with gray coloration can be found in the sequence list. Notably, putatively damaging variants were found in the genes LYST and ASIP, as well as regulatory variants in genes like MLPH, KIT, MITF, etc. Loss-of-function (LoF) variants in MLPH and both LoF variants and missense variants in LYST have been associated with gray coloration (aka coat color dilution) in several species including mice, dogs, cats, chicken, and cattle. ASIP on the other hand, is key regulator of MCIR and its loss of function has been shown to shift coat color from pheomelanin dominant (brown/red) to eumelanin dominant (black). In combination with disruption of MLPH and LYST, these edits should shift the coloration of the brown roan antelope (Hippotragus equinus) to gray.
Genome Editing: Cas9 and guide RNAs to generate coat color edits were delivered to roan antelope fibroblasts via chemical transfection using the CRISPRmax reagent (see methods). Editing efficiency and resulting variants were determined by next generation sequencing (NGS) on the Illumina MiSeq2. Insertions/deletions (indels) were generated as a consequence of double stranded break generation after Cas9-based cutting via non-homologous end joining. Indels were generated for the genes ASIP (FIGS. 1A and 1B), MLPH (FIGS. 2A and 2B), LYST (FIGS. 3A and 3B), and SLC45A2 (FIGS. 4A and 4B). Editing efficiencies were variable across loci, consistent with previous reports. These results demonstrated the ability to generate LoF variants in genes that generate a gray coat color.

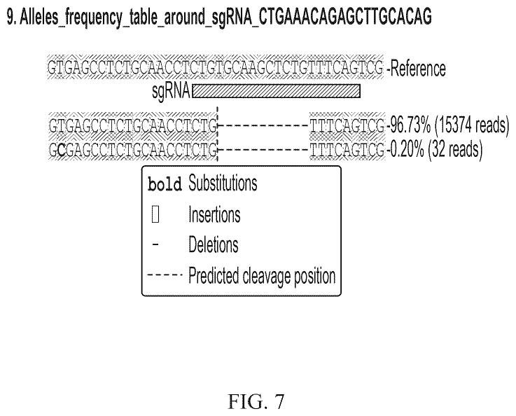

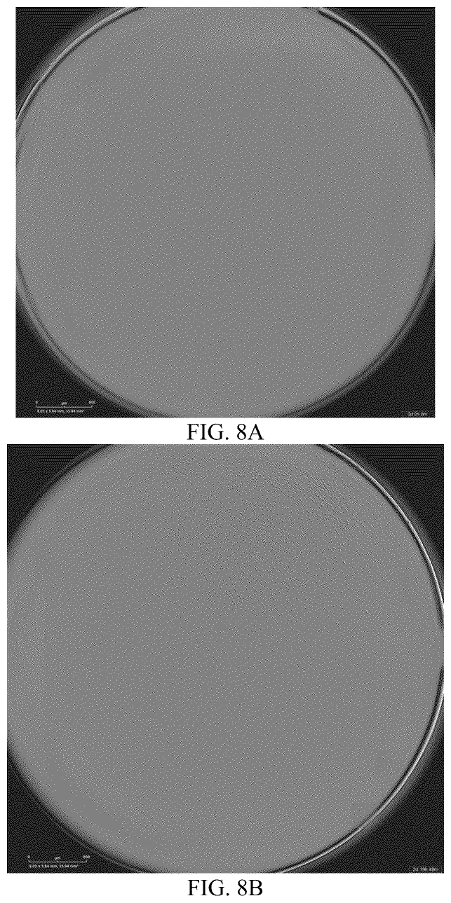

The ability to monoclonally isolate edited fibroblast cells was next demonstrated. After editing, single cells were sorted by FACS into individual wells of a 96-well plate. Cells were grown on conditioned media after sorting (see methods for more details) to enhance outgrowth. Genotyping via NGS revealed single colonies containing biallelic/homozygous edits in the LYST gene (FIG. 7). Colonies showed significant growth 7 days after monoclonal isolation (FIG. 8), indicating that cells remained healthy after the editing and isolation processes.

### Example 3: Creation of Roan Fibroblast Monoclonal Cells Lines Containing Triple Knock Out of MLPH and ASIP Genes with Cell Raft

Purpose: Generate phenotype engineering edits in monoclonal roan antelope cells to generate gray coat color in service of de-extincting the bluebuck antelope.

### Materials/Methods

Cell Raft Protocol: 1×DPBS (Mg2+ and Ca2+ free) was warmed to 37° C. The reservoir(s) were filled with warmed 1×DPBS. For a quad array 1 mL per reservoir was used. For either single 200 μm or 100 μm, 3 mL of 1× DPBS was used. The 1× DPBS was allowed to incubate for at least 3 minutes. The PBS was removed with either low pressure vacuum or P1000 by tilting the array to the side and aspirating on the corner of the pooled 1×PBS. The washing steps were repeated twice more. If coating was used, the coating was added and the array sat overnight at 37° C. The reservoirs were washed 2-3 times the next day with sterile DI water, and then culture media was added (for quad array, 500 μl per reservoir was added, and for single use, 3 μl per use was added). The media was allowed to incubate for at least 3 minutes while cells were being prepared.

Preparing Cell Suspension: The cells were counted. An appropriate number of cells was added to the volume needed in a separate tube. The media was removed from the array. The cell suspension was added with a P1000 dropwise onto the yellow-brown grid. The array was placed in a standard 37° C. incubator. When an array was moved, the array was picked up vertically and then transferred instead of sliding against surface. Either the top of the array was labelled such that the yellow-brown grid was not obscured, or the side of the array was labelled. The lot number and array specifications were recorded for downstream data input.

Scanning Arrays: 4 hours after seeding cells, the array was scanned to determine which wells had single cells using the AIR System (Cell Microsystems; Durham, NC).

Isolations: Warm media was gently added to the arrays. The data was analyzed, and the rafts of interest were mapped to isolate or create a gate for the system to automatically isolate from. 100 μl of respective warm media was placed in each well of a 96 well flat bottom tissue culture plate corresponding to the plate map or 5 μl of lysis buffer in each well of PCR plate. The AIR system was turned on, and the AIR software was launched. A lint-free wipe was sprayed with ethanol, and the interior of the AIR system was wiped down. The Isolation Plate Map was selected, and then, “Access Wand” and “Wand Access Position” was clicked. The wand was thoroughly cleaned with ethanol and then clicked into place. “Wand to Home” and “Return Home” were subsequently clicked. The array was then loaded onto the collection plate. The lids of the 96-well plate were removed. The door was closed, and “Isolate” was selected. Upon completion of the isolation, the lids were placed back on the array, and the plate was returned to the incubator.

Results: 171 monoclonal lines were generated from the initial isolation that grew at normal doubling rates 1 week post editing and isolation. Cell lines were genotyped and 12 cell lines were found to contain edits to all three genes targeted. Lines were biobanked for future experiments and use in editing.

### Example 4: Creation of Steel Coat Mouse

Purpose: Generated knockout edits to ASIP and MLPH in 129 mouse (agouti/brown coat color) ES cells to model proposed bluebuck phenotype engineering edits.

### Materials/Methods

### Reagents:

TABLE 2
Target sgRNA sequences for ASIP and MLPH

| sgRNA name | sgRNA sequence | SEQ ID NO |
|---|---|---|
| KO ASIP 1 | TAACTCCTCCATGAACTCGC | 256 |
| KO ASIP 3 | CTGGCACTCGAGGAGACGCT | 257 |
| KO MLPH 1 | GCTGTTGAGGGGCTGGTCTC | 258 |
| KO MLPH 2 | AGACAGTGAGCAGACTGATG | 259 |
| MLPH sgRNA Ch71 | CCTTTCCACGCTCACAGACG | 260 |

sgRNA for both ASIP and MLPH was combined with gene editing enzyme and transfected into N2A cell following IDT protocol using RNAiMAX. Briefly, sgRNA stock (synthesized by Synthego (Redwood City, CA), dissolved in TE to 100 μM) was further diluted to 1 μM in TE. The gene editing enzyme (Synthego SpCas9 2NLS nuclease 300 μmol, NC2245954) was diluted to 1 μM in OptiMem (Thermo Fisher; Waltham, MA). For RNP formation, 1.5 μl of sgRNA (1 μM), 1.5 μl of gene editing enzyme (1 μM), 22 μl OptiMem (total 25 μl), was mixed at room temperature (RT) for 5 minutes. After mixing, the solution was kept at 4° C.

At the same time, 1.2 μl RNAiMAX (Thermo Fisher) was diluted in 23.8 μl of OptiMem (total 25 μl). The RNAiMAX mixture was added to the RNP solution (total 50 μl). The mixture was incubated at RT for 20 minutes. During the 20 minute incubation, the cells were dissociated and were diluted to a concentration of 4×10⁵ cells/ml. 100 μl of the cells was used for each transfection (4×10⁴ cells). The cells were transfected with the 50 μl RNP/RNAiMAX, (final RNP=10 nM) mix for 2 days (no media change).

gDNA extraction: The cell media was removed, the cells were washed with PBS, and were lysed with 50 μl of quickExtract. The lysed cells were diluted with 100 μl of H₂O and 2 μl of the diluted sample was used as a template for PCR.

PCR condition: The PCR reaction mix was incubated at 98° C. for 20 seconds, then the following cycle was done 30 times: 98° C. for 10 seconds, 58° C. for 5 seconds, and 68° C. for 5 seconds; followed by 68° C. for 5 seconds.

T7E1 assay: For the T7E1 assay, 10 μl of PCR reaction was mixed with 8 μl H₂O and 2 μl of 10× Buffer 2. The digest was annealed with 1 μl of T7E1 at 37° C. for 20 minutes.

TABLE 3
PCR primer information

| Primer name | Sequence Information | SEQ ID NO |
|---|---|---|
| PCR ASIP F | TCATGTCCCCTTTGCTTCAATC | 261 |
| PCR ASIP R | CCAAGTCCACCAAGCATCTTGACT | 262 |
| PCR MLPH F | CTTCAAAGCCCTTGACATACCTCCT | 263 |
| PCR MLPH R | GGAATTGGGTAGGAGGGAGAAGC | 264 |

Cells: Neuro-2a cells are mouse neuroblasts with neuronal and amoeboid stem cell morphology isolated from the brain tissue of the mouse. The cells lines were thawed and resuspended in cDMEM (DMEM, Fetal Bovine Serum 10%, Pen/Strep) and plated in a T75 culture flask. At 90% confluency, the cells were split into four T75 culture flasks. Once the cells were confluent, three T75 culture flasks were frozen down, and one T75 culture flask was split 1:6.

The cells were electroporated with the RNP containing sgRNAs targeting mouse MLPH and ASIP. After electroporation, 750 cells were plated onto one well of a six-well plate (replicate of 3) for manual picking. The remainder of the cells were plated into 24-well plates (coated with a layer of mouse embryonic feeder cells prior to plating). 24 hours after electroporation, images were taken with a green filter to empirically assess transfection efficiency. 48 hours after electroporation, cells were pelleted down for DNA isolated and subsequent sequencing/T7 assay to determine cutting efficiency of the guides.

From the experiments performed, 30 clones for MLPH KO 2; 24 clones from MLPH KO 3; 48 clones from ASIP KO 2 and MLPH KO 2; and 48 clones from ASIP KO 3 and MLPH KO 2 were frozen down.

Sequencing of clones: From the frozen clones, the following three candidate clones were identified: (a) Clone A6, which contained a 1 base pair insertion in ASIP and a 2 base pair deletion in MLPH; (b) Clone B6, which contained a 1 base pair insertion in ASIP and a 1 base pair insertion in MLPH; and (c) Clone E1, which contained a 1 base pair insertion in ASIP and a 4 base pair deletion in MLPH.

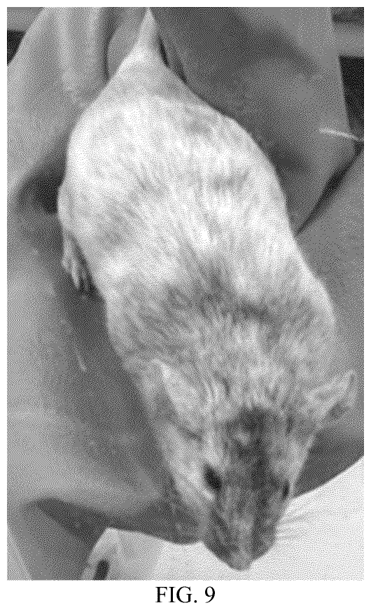

Results: Monoclonal embryonic stem cell(ES) lines were generated containing indel edits leading to total knockout of ASIP and MLPH. These cells were subsequently injected into albino embryos to generate chimeric mice (FIG. 9). The chimeric mice had gray patches on the fur demonstrating that the ASIP and MLPH edits resulted in the production of a gray coat color.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A transgenic animal comprising at least one bluebuck (Hippotragus leucophaeus) gene variant associated with gray/blue coat color and/or melanogenesis and/or at least one bluebuck variant associated with altered facial pattern.

2. The transgenic animal of claim 1, wherein;
(a) the at least one bluebuck gene variant associated with gray/blue coat color is selected from the group consisting of an ATP Binding Cassette Subfamily B Member 6 (LAN Blood Group) (ABCB6) variant, an Angiotensin Converting Enzyme 2 (ACE2) variant, an Adaptor Related Protein Complex 3 Subunit Beta 1 (AP3B1) variant, an Adaptor Related Protein Complex 3 Subunit Delta 1 (AP3D1) variant, an Agouti Signaling Protein (ASIP) variant, an ATPase Copper Transporting Alpha (ATP7A) variant, a BCL2 Apoptosis Regulator (BCL2) variant, a Biogenesis Of Lysosomal Organelles Complex 1 Subunit 2 (BLOC1S2) variant, a Biogenesis Of Lysosomal Organelles Complex 1 Subunit 3 (BLOC1S3) variant, a Biogenesis Of Lysosomal Organelles Complex 1 Subunit 4 (BLOC1S4) variant, a Biogenesis Of Lysosomal Organelles Complex 1 Subunit 5 (BLOC1S5) variant, a B-Raf Proto-Oncogene, Serine/Threonine Kinase (BRAF) variant, a Dopachrome Tautomerase (DCT) variant, a Dynactin Subunit 1 (DCTN1) variant, a Dynactin Subunit 2 (DCTN2) variant, a Dedicator Of Cytokinesis 7 (DOCK7) variant, a Dual Serine/Threonine And Tyrosine Protein Kinase (DSTYK) variant, a Dystrobrevin Binding Protein 1 (DTNBP1) variant, an Embryonic Ectoderm Development (EED) variant, a FIG. 4 Phosphoinositide 5-Phosphatase (FIG. 4) variant, a Gamma-Glutamyltransferase 1 (GGT1) variant, a G Protein-Coupled Receptor 143 (GPR143) variant, a HPS1 Biogenesis Of Lysosomal Organelles Complex 3 Subunit 1 (HPS1) variant, a HPS3 Biogenesis Of Lysosomal Organelles Complex 2 Subunit 1 (HPS3) variant, a HPS4 Biogenesis Of Lysosomal Organelles Complex 3 Subunit 2 (HPS4) variant, a HPS5 Biogenesis Of Lysosomal Organelles Complex 2 Subunit 2 (HPS5) variant, a HPS6 Biogenesis Of Lysosomal Organelles Complex 2 Subunit 3 (HPS6) variant, a Lysosomal Trafficking Regulator (LYST) variant, a Membrane Bound Transcription Factor Peptidase, Site 1 (MBTPS1) variant, a Mucolipin TRP Cation Channel 3 (MCOLN3) variant, a Major Facilitator Superfamily Domain Containing 12 (MFSD12) variant, a Melanocyte Inducing Transcription Factor (MITF) variant, a Muskelin 1 (MKLN1) variant, a Melan-A (MLANA) variant, a Melanophilin (MLPH) variant, a Melanoregulin (MREG) variant, a Myosin VA (MYO5A) variant, a Notch Receptor 1 (NOTCH1) variant, a Notch Receptor 2 (NOTCH2) variant, an OCA2 Melanosomal Transmembrane Protein (OCA2) variant, a Phenylalanine Hydroxylase (PAH) variant, a Phosphoinositide Kinase, FYVE-Type Zinc Finger Containing (PIKFYVE) variant, a Premelanosome Protein (PMEL) variant, a RAB27A, Member RAS Oncogene Family (RAB27A) variant, a RAB38, Member RAS Oncogene Family (RAB38) variant, a Rab Geranylgeranyltransferase Subunit Alpha (RABGGTA) variant, a Raf-1 Proto-Oncogene, Serine/Threonine Kinase (RAF1) variant, a Solute Carrier Family 24 Member 5 (SLC24A5) variant, a Solute Carrier Family 45 Member 2 (SLC45A2) variant, a Solute Carrier Family 7 Member 11 (SLC7A11) variant, a Snail Family Transcriptional Repressor 2 (SNAI2) variant, a Sperm Associated Antigen 9 (SPAG9) variant, a Two Pore Segment Channel 2 (TPCN2) variant, a Tyrosinase (TYR) variant, a Tyrosinase Related Protein 1 (TYRP1) variant, a VAC14 Component Of PIKFYVE Complex (VAC14) variant, a VPS33A Core Subunit Of CORVET And HOPS Complexes (VPS33A) variant, a YKT6 V-SNARE Homolog (YKT6) variant, and combinations thereof;
(b) the at least one bluebuck gene variant associated with melanogenesis is selected from the group consisting of an Adaptor Related Protein Complex 3 Subunit Delta 1 (AP3D1) variant, an Activating Signal Cointegrator 1 Complex Subunit 2 (ASCC2) variant, an ATPase H+ Transporting V0 Subunit A2 (ATP6V0A2) variant, a CCDC53 variant, a CD72 molecule (CD72) variant, a Cyclin Dependent Kinase 12 (CDK12) variant, a Chromodomain Helicase DNA Binding Protein 8 (CHD8) variant, a COMM Domain Containing 5 (COMMD5) variant, a DSCR3 variant, an E1A Binding Protein P300 (EP300) variant, a FIG. 4 Phosphoinositide 5-Phosphatase (FIG. 4) variant, a FRY Like Transcription Coactivator (FRYL) variant, a Histone Deacetylase 4 (HDAC4) variant, a Heterogeneous Nuclear Ribonucleoprotein A2/B1 Like 1 (HNRNPA2B1L1) variant, a Heterogeneous Nuclear Ribonucleoprotein A2/B1 Like 2 (HNRNPA2B1L2) variant, a Heterogeneous Nuclear Ribonucleoprotein A2/B1 Like 3 (HNRNPA2B1L3) variant, a Heterogeneous Nuclear Ribonucleoprotein A2/B1 Like 4 (HNRNPA2B1L4) variant, a HPS3 Biogenesis Of Lysosomal Organelles Complex 2 Subunit 1 (HPS3) variant, a HPS4 Biogenesis Of Lysosomal Organelles Complex 3 Subunit 2 (HPS4) variant, a HPS5 Biogenesis Of Lysosomal Organelles Complex 2 Subunit 2 (HPS5) variant, a HPS6 Biogenesis Of Lysosomal Organelles Complex 2 Subunit 3 (HPS6) variant, an Integrator Complex Subunit 8 (INTS8) variant, a Lysosomal Trafficking Regulator (LYST) variant, a Nuclear Receptor Corepressor 1 (NCOR1) variant, a RAB9A, Member RAS Oncogene Family (RAB9A) variant, a RB Transcriptional Corepressor 1 (RB1) variant, a SR-Related CTD Associated Factor 4 (SCAF4) variant, a SEC31 Homolog A, COPII Coat Complex Component (SEC31A) variant, a Siah E3 Ubiquitin Protein Ligase 1 (SIAH1) variant, a SMG7 Nonsense Mediated MRNA Decay Factor (SMG7) variant, a Sorting Nexin 13 Like 1 (SNX13L1) variant, a Sorting Nexin 13 Like 2 (SNX13L2) variant, a TBC1 Domain Family Member 5 (TBC1D5) variant, a WD Repeat And FYVE Domain Containing 1 (WDFY1) variant, and combinations thereof; and/or
(c) the bluebuck gene variant associated with altered facial pattern is selected from the group consisting of an ADAM Metallopeptidase With Thrombospondin Type 1 Motif 20 (ADAMTS20) variant, a Bone Morphogenetic Protein 4 (BMP4) variant, a Dedicator Of Cytokinesis 7 (DOCK7) variant, an Endothelin 3 (EDN3) variant, an Endothelin Receptor Type B (EDNRB) variant, an ETS Proto-Oncogene 1, Transcription Factor Like 1 (ETS1L1) variant, an ETS Proto-Oncogene 1, Transcription Factor Like 2 (ETS1L2) variant, a Forkhead Box D3 (FOXD3) variant, a Histone Deacetylase 1 (HDAC1) variant, an Interferon Regulatory Factor 1 (IRF1) variant, a KIT Proto-Oncogene, Receptor Tyrosine Kinase (KIT) variant, a KIT Ligand (KITLG) variant, a Mucolipin TRP Cation Channel 3 (MCOLN3) variant, a Melanocyte Inducing Transcription Factor (MITF) variant, a Paired Box 3 (PAX3) variant, a Snail Family Transcriptional Repressor 2 (SNAI2) variant, a SRY-Box Transcription Factor 10 (SOX10) variant, a SRY-Box Transcription Factor 2 (SOX2) variant, a Transient Receptor Potential Cation Channel Subfamily M Member 1 (TRPM1) variant, a Wnt Family Member 3A (WNT3A) variant, a Zinc Finger Homeobox 4 (ZFHX4) variant, and combinations thereof.

3-4. (canceled)

5. The transgenic animal of claim 1, wherein the bluebuck gene variant comprises at least one change in the nucleotide sequence of the gene.

6. The transgenic animal of claim 5, wherein the change in the nucleotide sequence is a substitution, an insertion, a deletion, or a combination thereof.

7. The transgenic animal of claim 6, wherein the substitution, the insertion, the deletion, or a combination thereof is in a 5′ untranslated region of the gene, an intron of the gene, an exon of the gene, a 3′ untranslated region of the gene, a regulatory region of the gene, or a combination thereof.

8. (canceled)

9. The transgenic animal of claim 2, wherein;
(i) the bluebuck gene variant associated with gray/blue coat color comprises: (a) a nucleotide sequence having at least 80%, at least 85%, at least 90%, or at least 95% identity to a nucleotide sequence selected from the group consisting of SEQ ID NOs: 45-102 and combinations thereof; or (b) a nucleotide sequence selected from the group consisting of SEQ ID NOs: 147-204, and combinations thereof;
(ii) the bluebuck gene variant associated with melanogenesis comprises: (a) a nucleotide sequence having at least 80%, at least 85%, at least 90%, or at least 95% identity to a nucleotide sequence selected from the group consisting of SEQ ID NOs: 48, 64, 68-72, 120-146, and combinations thereof; or (b) a nucleotide sequence selected from the group consisting of SEQ ID NOs: 150, 166, 170-174, 222-248, and combinations thereof; and/or
(iii) the bluebuck gene variant associated with altered facial pattern comprises: (a) a nucleotide sequence having at least 80%, at least 85%, at least 90%, or at least 95% identity to a nucleotide sequence selected from the group consisting of SEQ ID NOs: 60, 74, 76, 95, 103-119, and combinations thereof; or (b) comprises a nucleotide sequence selected from the group consisting of SEQ ID NOs: 162, 176, 178, 197, 205-221, and combinations thereof.

10-14. (canceled)

15. The transgenic animal of claim 1, wherein the transgenic animal comprises at least 2, 3, 4, 5, 6, 7, 8, 9, 10, 15, 20, 25, 30, 40, 50, 60, 70, 80, 90, 95, 100, 101, 102 (or any number in between) bluebuck gene variants.

16. The transgenic animal of claim 1, wherein the transgenic animal further comprises at least one bluebuck gene variant associated with gray/blue coat color selected from the group consisting of:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.

17. A transgenic animal comprising at least one bluebuck (Hippotragus leucophaeus) gene variant associated with gray/blue coat color selected from the group consisting of:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.

18. The transgenic animal of claim 1, wherein the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

19. The transgenic animal of claim 18, wherein the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RBI, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

20. The transgenic animal of claim 1, wherein the transgenic animal is an antelope.

21. The transgenic animal of claim 20, wherein the transgenic animal is a roan antelope (Hippotragus equinus) or a sable antelope (Hippotragus niger).

22. A recombinant host cell comprising at least one bluebuck (Hippotragus leucophaeus) gene variant associated with gray/blue coat color and/or melanogenesis and/or at least one bluebuck variant associated with altered facial pattern.

23. The recombinant host cell of claim 22, wherein;
(a) the at least one bluebuck gene variant associated with gray/blue coat color and/or melanogenesis is selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MLPH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, a YKT6 variant, an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SNX13L2 variant, a TBC1D5 variant, a WDFY1 variant, and combinations thereof; and/or
(b) the at least one bluebuck gene variant associated with altered facial pattern is selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

24. (canceled)

25. The recombinant host cell of claim 22, wherein the bluebuck gene variant comprises at least one change in the nucleotide sequence of the gene.

26. The recombinant host cell of claim 25, wherein the change in the nucleotide sequence is a substitution, an insertion, a deletion, or a combination thereof.

27. The recombinant host cell of claim 26, wherein the substitution, the insertion, the deletion, or a combination thereof is in a 5′ untranslated region of the gene, an intron of the gene, an exon of the gene, a 3′ untranslated region of the gene, a regulatory region of the gene, or a combination thereof.

28. (canceled)

29. The recombinant host cell of claim 23, wherein:
(i) the bluebuck gene variant associated with gray/blue coat color and/or melanogenesis comprises:
(a) a nucleotide sequence having at least 80%, at least 85%, at least 90%, or at least 95% identity to a nucleotide sequence selected from the group consisting of SEQ ID NOs: 45-102, 120-146, and combinations thereof; or
(b) nucleotide sequence selected from the group consisting of SEQ ID NOs: 147-204, 222-248, and combinations thereof; and/or
(ii) the bluebuck gene variant associated with altered facial pattern comprises:
(a) a nucleotide sequence having at least 80%, at least 85%, at least 90%, or at least 95% identity to a nucleotide sequence selected from the group consisting of SEQ ID NOs: 60, 74, 76, 95, 103-119, and combinations thereof; or
(b) a nucleotide sequence selected from the group consisting of SEQ ID NOs: 162, 176, 178, 197, 205-221, and combinations thereof.

30-32. (canceled)

33. The recombinant host cell of claim 22, wherein the recombinant host cell comprises at least 2, 3, 4, 5, 6, 7, 8, 9, 10, 15, 20, 25, 30, 40, 50, 60, 70, 80, 90, 95, 100, 101, 102 (or any number in between) bluebuck gene variants.

34. The recombinant host cell of claim 22, wherein the recombinant host cell further comprises at least one bluebuck gene variant associate with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.

35. A recombinant host cell comprising at least one bluebuck (Hippotragus leucophaeus) gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.

36. The recombinant host cell of claim 22, wherein the recombinant host cell fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

37. The recombinant host cell of claim 35, wherein the recombinant host cell fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

38. The recombinant host cell of claim 22, wherein the recombinant host cell is an antelope cell.

39. The recombinant host cell of claim 38, wherein the recombinant host cell is a roan antelope (Hippotragus equinus) cell or a sable antelope (Hippotragus niger) cell.

40. The recombinant host cell of claim 22, wherein the recombinant host cell is a stem cell, a reprogrammed cell, a fibroblast cell, a mesenchymal cell, endothelial progenitor cell (EPC), a pericyte, a nerve cell, a cartilage cell, a bone cell, a muscle cell, a fat cell, or an epidermal cell.

41. The recombinant host cell of claim 40, wherein the stem cell is selected from an induced stem cell, an embryonic stem (ES) cell, or a mesenchymal stem cell (MSC).

42-45. (canceled)

46. A method of creating a transgenic animal with gray/blue coat color and/or an altered melanin biosynthesis pathway of a bluebuck and/or an altered facial pattern of a bluebuck, the method comprising:
(a) obtaining a cell from the animal;
(b) introducing into the cell at least one bluebuck (Hippotragus leucophaeus) gene variant associated with gray/blue coat color and/or melanogenesis, wherein the one or more bluebuck gene variants associated with gray/blue coat color are selected from an ABCB6 variant, an ACE2 variant, an AP3B1 variant, an AP3D1 variant, an ASIP variant, an ATP7A variant, a BCL2 variant, a BLOC1S2 variant, a BLOC1S3 variant, a BLOC1S4 variant, a BLOC1S5 variant, a BRAF variant, a DCT variant, a DCTN1 variant, a DCTN2 variant, a DOCK7 variant, a DSTYK variant, a DTNBP1 variant, an EED variant, a FIG. 4 variant, a GGT1 variant, a GPR143 variant, a HPS1 variant, a HPS3 variant, a HPS4 variant, a HPS5 variant, a HPS6 variant, a LYST variant, a MBTPS1 variant, a MCOLN3 variant, a MFSD12 variant, a MITF variant, a MKLN1 variant, a MLANA variant, a MLPH variant, a MREG variant, a MYO5A variant, a NOTCH1 variant, a NOTCH2 variant, an OCA2 variant, a PAH variant, a PIKFYVE variant, a PMEL variant, a RAB27A variant, a RAB38 variant, a RABGGTA variant, a RAF1 variant, a SLC24A5 variant, a SLC45A2 variant, a SLC7A11 variant, a SNAI2 variant, a SPAG9 variant, a TPCN2 variant, a TYR variant, a TYRP1 variant, a VAC14 variant, a VPS33A variant, and a YKT6 variant, and the one or more bluebuck gene variants associated with melanogenesis are selected from an AP3D1 variant, an ASCC2 variant, an ATP6V0A2 variant, a CCDC53 variant, a CD72 variant, a CDK12 variant, a CHD8 variant, a COMMD5 variant, a DSCR3 variant, an EP300 variant, a FIG. 4 variant, a FRYL variant, a HDAC4 variant, a HNRNPA2B1L1 variant, a HNRNPA2B1L2 variant, a HNRNPA2B1L3 variant, a HNRNPA2B1L4 variant, a HPS3 variant, a HPS4 variant, a HPS5 variant, a HPS6 variant, an INTS8 variant, a LYST variant, a NCOR1 variant, a RAB9A variant, a RB1 variant, a SCAF4 variant, a SEC31A variant, a SIAH1 variant, a SMG7 variant, a SNX13L1 variant, a SLX13L2 variant, a TBC1D5 variant, and a WDFY1 variant; and/or the bluebuck gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant; and
(c) utilizing the recombinant cell to produce a transgenic animal,
wherein the transgenic animal has a gray/blue coat color and/or an altered melanin biosynthesis pathway of a bluebuck and/or an altered facial pattern of a bluebuck.

47. The method of claim 46, wherein the bluebuck gene variant comprises at least one change in the nucleotide sequence of the gene.

48. The method of claim 47, wherein the change in the nucleotide sequence is a substitution, an insertion, a deletion, or a combination thereof.

49. The method of claim 48, wherein the substitution, the insertion, the deletion, or a combination thereof is in a 5′ untranslated region of the gene, an intron of the gene, an exon of the gene, a 3′ untranslated region of the gene, regulatory region of the gene, or a combination thereof.

50. (canceled)

51. The method of claim 46, wherein:
(i) the bluebuck gene variant associated with gray/blue coat color and/or melanogenesis comprises:
(a) a nucleotide sequence having at least 80%, at least 85%, at least 90%, or at least 95% identity to a nucleotide sequence selected from the group consisting of SEQ ID NOs: 45-102, 120-146, and combinations thereof; or
(b) a nucleotide sequence selected from the group consisting of SEQ ID NOs: 147-204, 222-248, and combinations thereof; and/or
(ii) the bluebuck gene variant associated with altered facial pattern comprises:
(a) a nucleotide sequence having at least 80%, at least 85%, at least 90%, or at least 95% identity to a nucleotide sequence selected from the group consisting of SEQ ID NOs: 60, 74, 76, 95, 103-119, and combinations thereof; or
(b) a nucleotide sequence selected from the group consisting of SEQ ID NOs: 162, 176, 178, 197, 205-221, and combinations thereof.

52-54. (canceled)

55. The method of claim 46, wherein at least 2, 3, 4, 5, 6, 7, 8, 9, 10, 15, 20, 25, 30, 40, 50, 60, 70, 80, 90, 95, 100, 101, 102 (or any number in between) bluebuck gene variants are introduced into the cell.

56. The method of claim 46, wherein the transgenic animal further comprises at least one bluebuck gene variant associated with gray/blue coat color selected from:
(a) a loss-of-function agouti signaling protein (ASIP) variant;
(b) a defensin beta 103 (DEFB103) missense variant;
(c) a loss-of-function melanophilin (MLPH) variant;
(d) a premelanosome protein (PMEL) missense variant;
(e) a loss-of-function lysosomal trafficking regulator (LYST) variant; and/or
(f) a loss-of-function solute carrier family 45 member 2 (SLC45A2) variant.

57. A method of creating a transgenic animal with gray/blue coat color, the method comprising:
(a) obtaining a cell from the animal;
(b) introducing into the cell at least one bluebuck gene variant associated with gray/blue coat color selected from:
(1) a loss-of-function agouti signaling protein (ASIP) variant;
(2) a defensin beta 103 (DEFB103) missense variant;
(3) a loss-of-function melanophilin (MLPH) variant;
(4) a premelanosome protein (PMEL) missense variant;
(5) a loss-of-function lysosomal trafficking regulator (LYST) variant; and/or
(6) a loss-of-function solute carrier family 45 member 2 (SLC45A2) variant, whereby introducing the at least one bluebuck gene variant produces a recombinant cell; and
(c) utilizing the recombinant cell to produce a transgenic animal,
wherein the transgenic animal has a gray/blue coat color.

58. The method of claim 57, wherein the transgenic animal further comprises at least one bluebuck (Hippotragus leucophaeus) gene variant associated with altered facial pattern selected from an ADAMTS20 variant, a BMP4 variant, a DOCK7 variant, an EDN3 variant, an EDNRB variant, an ETS1L1 variant, an ETS1L2 variant, a FOXD3 variant, a HDAC1 variant, an IRF1 variant, a KIT variant, a KITLG variant, a MCOLN3 variant, a MITF variant, a PAX3 variant, a SNAI2 variant, a SOX10 variant, a SOX2 variant, a TRPM1 variant, a WNT3A variant, and a ZFHX4 variant.

59. The method of claim 46, wherein the transgenic animal fails to express an endogenous homologue of at least one of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

60. The method of claim 46, wherein the transgenic animal fails to express the endogenous homologue of 2, 3, 4, 5, 6, 7, 8, 9, 10, 15, 20, 25, 30, 35, 40, 45, 50, 55, 60, 65, 70, 75, 80, 85, 90, 95, 100, 101, 102 (or any number in between) of ABCB6, ACE2, AP3B1, AP3D1, ASIP, ATP7A, BCL2, BLOC1S2, BLOC1S3, BLOC1S4, BLOC1S5, BRAF, DCT, DCTN1, DCTN2, DOCK7, DSTYK, DTNBP1, EED, FIG. 4, GGT1, GPR143, HPS1, HPS3, HPS4, HPS5, HPS6, LYST, MBTPS1, MCOLN3, MFSD12, MITF, MKLN1, MLANA, MLPH, MREG, MYO5A, NOTCH1, NOTCH2, OCA2, PAH, PIKFYVE, PMEL, RAB27A, RAB38, RABGGTA, RAF1, SLC24A5, SLC45A2, SLC7A11, SNAI2, SPAG9, TPCN2, TYR, TYRP1, VAC14, VPS33A, YKT6, ASCC2, ATP6V0A2, CCDC53, CD72, CDK12, CHD8, COMMD5, DSCR3, EP300, FRYL, HDAC4, HNRNPA2B1L1, HNRNPA2B1L2, HNRNPA2B1L3, HNRNPA2B1L4, INTS8, NCOR1, RAB9A, RB1, SCAF4, SEC31A, SIAH1, SMG7, SNX13L1, SNX13L2, TBC1D5, WDFY1, ADAMTS20, BMP4, EDN3, EDNRB, ETS1L1, ETS1L2, FOXD3, HDAC1, IRF1, KIT, KITLG, PAX3, SOX10, SOX2, TRPM1, WNT3A, and ZFHX4.

61. The method of claim 46, wherein the cell is a stem cell, a reprogrammed cell, a fibroblast cell, a mesenchymal cell, a nerve cell, a cartilage cell, a bone cell, a muscle cell, a fat cell, an epidermal cell, an endothelial progenitor cell (EPC), or a pericyte.

62. The method of claim 61, wherein the stem cell is selected from an induced stem cell, an embryonic stem (ES) cell, or a mesenchymal stem cell (MSC).

63-66. (canceled)

67. The method of claim 46, wherein the animal is an antelope.

68. The method of claim 67, wherein the antelope is selected from a roan antelope (Hippotragus equinus) or a sable antelope (Hippotragus niger).

69. A transgenic animal made by the method claim 46.
